(19) United States
(12) Patent Application Publication
(10) Pub. No.: US 2025/0049724 A1
(43) Pub. Date: Feb. 13, 2025

(54) MICROCAPSULE DISPERSIONS WITH EMULSIFIER

(71) Applicant: KOEHLER INNOVATION & TECHNOLOGY GMBH, Oberkirch (DE)

(72) Inventors: Andreas Bauer, Kaarst (DE); Marc-Steffen SCHIEDEL, Monheim (DE); Stefan URLICHS, Essen (DE); Michael BIEDENBACH, Oberkirch (DE); Christian KÖLMEL, Baden-Baden (DE)

(73) Assignee: KOEHLER INNOVATION & TECHNOLOGY GMBH, Oberkirch (DE)

(21) Appl. No.: 18/718,952

(22) PCT Filed: Dec. 14, 2022
(86) PCT No.: PCT/DE2022/200297

§ 371 Date: Jun. 12, 2024

(30) Foreign Application Priority Data
Dec. 15, 2021 (DE) 10 2021 214 457.8

(51) Int. Cl. A61K 9/48; A61K 9/107; C09D 5/02

(52) U.S. Cl. CPC A61K 9/4808; A61K 9/107; A61K 9/4816; C09D 5/022

(57) ABSTRACT

A microcapsule dispersion containing:
(1) biodegradable microcapsules comprising a core material and a shell, wherein the shell consists of at least one barrier layer and one stability layer, wherein the barrier layer surrounds the core material, wherein the stability layer comprises at least one biopolymer and is arranged on the outer surface of the barrier layer, and wherein optionally an emulsion stabilizer is arranged at the transition from the barrier layer to the stability layer; and
(2) at least one emulsifier, wherein the emulsifier is selected from the group of ethoxylated, hydrogenated castor oils, particularly those with medium EO values in the range of 20 to 60, preferably 30 to 50.

### FIELD OF THE INVENTION

The invention relates to microcapsule dispersions containing biodegradable microcapsules with environmentally friendly wall materials as well as particular emulsifiers.

### BACKGROUND OF THE INVENTION

Microencapsulation is a versatile technology. It offers solutions for numerous innovations—from the paper industry to household products, microencapsulation enhances the functionality of various active substances. Encapsulated active ingredients can be used more economically, improving the sustainability and environmental compatibility of many products.

However, the polymeric wall materials of the microcapsules themselves are environmentally compatible to very different degrees. Microcapsule walls based on the natural product gelatin and thus completely biodegradable have long been used in carbonless paper. A gelatin encapsulation process developed as early as the 1950s is disclosed in U.S. Pat. No. 2,800,457. Since then, a variety of variations in terms of materials and process steps have been described. In addition, biodegradable or enzymatically degradable microcapsule walls are used to employ enzymatic degradation as a method for releasing the core material. Such microcapsules are described, for example, in WO 2009/126742 A1 or WO 2015/014628 A1.

However, such microcapsules are not suitable for many industrial applications and household products. This is because natural-substance-based microcapsules do not meet the diffusion impermeability, chemical resistance and temperature resistance required for detergents and cleaning agents, adhesive systems, coatings and dispersions, for example, nor the required loading with core material.

In these so-called high-demand areas, classic organic polymers such as melamine-formaldehyde polymers (see, e.g., EP 2 689 835 A1, WO 2018/114056 A1, WO 2014/016395 A1, WO 2011/075425 A1, or WO 2011/120772 A1); polyacrylates (see, e.g., WO 2014/032920 A1, WO 2010/79466 A2); polyamides; polyurethane or polyurea (see, e.g., WO 2014/036082 A2 or WO 2017/143174 A1) are used. Capsules made from such organic polymers have the necessary diffusion impermeability, stability, and chemical resistance. However, these organic polymers are only minimally enzymatically or biologically degradable.

In the state of the art, various approaches are described in which biopolymers are combined as an additional component with the organic polymers of the microcapsule shell for use in high-demand areas. However, this is not with the goal of producing biodegradable microcapsules, but primarily to alter the release, stability, or surface properties of the microcapsules. For example, in WO 2014/044840 A1, a method for producing two-layered microcapsules described with an inner polyurea layer and an outer gelatin-containing layer. The polyurea layer is generated by polyaddition on the inside of the gelatin layer obtaining by coacervation. The capsules thus obtained have the necessary stability and impermeability for use in detergents and cleaning agents due to the polyurea layer and additionally by the gelatin to attach them to surfaces. Concrete stabilities and resistance are not mentioned. However, a disadvantage of polyurea capsules is the inevitable side reaction of the core materials with the diisocyanates used to produce the urea, which must be added to the oil-based core.

On the other hand, biopolymer-based microcapsules are also described in the prior art, which achieve improved impermeability or stability against environmental influences or a targeted setting of a delayed release behavior by adding a protective layer. For example, WO 2010/003762 A1 describes particles with a core-shell-shell structure. Inside each particle there is a poorly water-soluble or water-insoluble organic active ingredient as a core. The shell directly surrounding the core contains a biodegradable polymer and the outer shell contains at least one metal or semimetal oxide. With this structure, a biodegradable shell is indeed obtained. Nevertheless, the microcapsules are used in food, cosmetics or pharmaceuticals according to WO 2010/003762 A1, but are not usable for the high-demand areas according to the invention due to lack of impermeability.

Unpublished PCT/EP2020/085804 describes microcapsules with a multilayer shell structure that are essentially biodegradable and still provide sufficient stability and tightness for use in high-demand areas. This is achieved by a stability layer forming the main part of the capsule shell, consisting of naturally occurring and highly biodegradable materials, such as gelatin or alginate, or from materials ubiquitously present in nature.

This stability layer is combined with a barrier layer, which may consist of known materials used in microencapsulation, such as melamine-formaldehyde or meth(acrylate). It has been possible to design the barrier layer with a previously unachievable low wall thickness while still ensuring sufficient impermeability. As a result, the proportion of the barrier layer in the total wall is kept very low, so that the microcapsule wall exhibits a biodegradability measured according to OECD 301 F of at least 40%.

Such microcapsules are typically used in the form of aqueous suspensions, also referred to as slurries, in which the microcapsules are dispersed as a solid phase in a predominantly aqueous medium serving as the continuous phase. It is desirable for such suspensions to exhibit sufficient phase stability to provide a stable product without unwanted sedimentation or creaming, even after extended storage or transport times.

For this purpose, various additives or auxiliaries are often incorporated into the continuous phase to ensure this stability. However, these must often be specifically selected depending on the capsules used, as general suitability is typically not given.

The present invention relates to microcapsule dispersions in which a particular emulsifier is used that provides the desired phase stability in the microcapsule dispersion and a final product containing the microcapsules.

### SUMMARY OF THE INVENTION

According to a first aspect, the invention relates to a microcapsule dispersion containing:
(1) biodegradable microcapsules comprising a core material and a shell, wherein the shell consists of at least one barrier layer and one stability layer, wherein the barrier layer surrounds the core material, wherein the stability layer comprises at least one biopolymer and is arranged on the outer surface of the barrier layer, and wherein optionally an emulsion stabilizer is arranged at the transition from the barrier layer to the stability layer; and
(2) at least one emulsifier, wherein the emulsifier is selected from the group of ethoxylated, hydrogenated castor oils, particularly those with medium EO values in the range of 20 to 60, preferably 30 to 50.

In various embodiments of the invention, the emulsifier is used as a component of a microcapsule dispersion (slurry), wherein the suspension comprises the microcapsules as a solid phase and water as the main component of the continuous phase. In such embodiments, the emulsifier is part of the continuous phase.

In various embodiments, the barrier layer and the stability layer differ in their chemical composition or their chemical structure. The core material preferably comprises at least one fragrance and can, for example, be a perfume oil composition.

The emulsion stabilizer is a polymer or copolymer of certain acrylic acid derivatives, N-vinylpyrrolidone, and/or styrene. In various embodiments, the polymer or copolymer consists of one or more monomers selected from:
(1) Acrylic acid derivatives of the general formula (I)

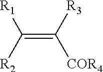

wherein
R₁, R₂ and R₃ are selected from hydrogen and an alkyl group with 1 to 4 carbon atoms, wherein R₁ and R₂ are particularly hydrogen and R₃ is particularly hydrogen or methyl; and
R₄ is for —OX or —NR₅R₆, wherein X is hydrogen, an alkali metal, an ammonium group, or a C₁-C₁₈ alkyl possibly substituted by —SO₃M or —OH, wherein M is hydrogen, an alkali metal, or ammonium, wherein the optionally —SO₃M or —OH substituted C₁-C₁₈ alkyl is preferably methyl, ethyl, n-butyl, 2-ethylhexyl, 2-sulfoethyl, or 2-sulfopropyl, wherein R₅ and R₆ are independently selected from hydrogen or a C₁-C₁₀ alkyl possibly substituted by —SO₃M, wherein at least one of R₅ and R₆ is not hydrogen, and wherein preferably R₅ is H and R₆ is 2-methyl-propan-2-yl-1-sulfonic acid;
(2) N-vinylpyrrolidone; and
(3) styrene.

The emulsion stabilizer is preferably an acrylate copolymer containing 2-acrylamido-2-methylpropanesulfonic acid (AMPS). A suitable copolymer is available, for example, under the trade name Dimension PA 140.

In various embodiments, the barrier layer is composed of one or more components selected from the group consisting of an aldehydic component, an aromatic alcohol, an amine component, an acrylate component, and an isocyanate component, and the stability layer comprises at least one biopolymer.

Furthermore, it is advantageous that the improved structural integration of the stability layer by the barrier layer, through the addition of the emulsion stabilizer, ensures the structural (covalent) connection of all wall-forming components, so that the individual layers are inseparably connected and can be considered as a monopolymer.

Due to the robustness and impermeability of the biodegradable capsules, they can be used in a variety of products in the fields of detergents and cleaning agents, as well as cosmetics.

Furthermore, the invention relates, in a further aspect, to the use of the microcapsule dispersion according to the first aspect for the production of a product, wherein the microcapsule dispersion is used to produce the product or its intermediate product, and wherein the final product or the intermediate product has a pH value of less than 11, preferably less than 9, more preferably less than 5, and most preferably less than 4 and/or a conductivity of at least 0.1 mS/cm, preferably at least 0.2 mS/cm, and at most 100 mS/cm, preferably up to 60 mS/cm, and most preferably 34 mS/cm.

Furthermore, the invention relates, in a further aspect, to a product containing a microcapsule dispersion according to the first aspect, with a pH value of less than 11, preferably less than 9, more preferably less than 5, and most preferably less than 4 and/or a conductivity of more than 0.3 mS/cm, preferably more than 1.0 mS/cm, more preferably more than 2.5 mS/cm, and most preferably more than 5.0 mS/cm.

### DETAILED DESCRIPTION OF THE INVENTION

### Definitions

“Barrier layer” refers to the layer of a microcapsule wall that is essentially responsible for the impermeability of the capsule shell, i.e., it prevents the release of the core material.

“Biodegradability” refers to the ability of organic chemicals to be biologically decomposed, that is, by living organisms or their enzymes. Ideally, this chemical metabolism proceeds completely to mineralization, but may stop at degradation-stable transformation products. Generally accepted are the guidelines for testing chemicals of the OECD, which are also used in the context of chemical approval. The tests of the OECD Test Series 301 (A-F) demonstrate rapid and complete biodegradation (ready biodegradability) under aerobic conditions. Different test methods are available for well or poorly soluble as well as for volatile substances. In particular, the manometric respiration test (OECD 301 F) is used within the scope of the application. The basic biodegradability (inherent biodegradability) can be determined via the measurement standard OECD 302, for example the MITI II test (OECD 302 C).

“Biodegradable” or “biologically degradable” in the sense of the present invention refers to microcapsule walls that exhibit a biodegradability, measured according to OECD 301 F, of at least 40% within 60 days. Microcapsule walls that show a degradation of at least 60% within 60 days, measured according to OECD 301 F, are also referred to here as rapidly biodegradable.

A “biopolymer” is a naturally occurring polymer, for example, a polymer found in a plant, fungus, bacterium, or animal. Biopolymers also include modified polymers based on naturally occurring polymers. The biopolymer can be derived from the natural source or synthetically produced.

“Impermeability” to a substance, gas, liquid, radiation or the like, is a property of material structures. According to the invention, the terms “impermeability” and “tightness” are used synonymously. Impermeability is a relative term and always refers to given general conditions.

“Emulsion stabilizers” are auxiliaries used for stabilizing emulsions. The emulsion stabilizers can be added in small amounts to the aqueous or oily phase (of emulsions) where they become enriched at the interface in a phase-oriented manner, thereby facilitating the dispersion of the internal phase by reducing the interfacial tension and increasing the resistance to dispersion of the emulsion.

The term “(meth)acrylate” in the present invention refers to both methacrylates and acrylates.

According to the invention, the term “microcapsules” refers to particles containing an inner space or core filled with a solid, gelled, liquid or gaseous medium and enclosed (encapsulated) by a continuous coating (shell) of film-forming polymers. These particles preferably have small dimensions. The terms “microcapsules”, “core-shell capsules”, or simply “capsules” are used interchangeably.

“Microencapsulation” refers to a manufacturing process in which small and miniscule portions of solid, liquid or gaseous substances are surrounded by a shell of polymeric or inorganic wall materials. The microcapsules thus obtained may have a diameter of a few millimeters to less than 1 μm.

The microcapsule features a multi-layered “shell.” The shell enclosing the core material of the microcapsule is also regularly referred to as the “wall” or “sheath”.

The microcapsules with a multi-layered shell can also be referred to as multi-layered microcapsules or a multi-layered microcapsule system, as the individual layers can also be regarded as individual shells. “Multi-layered” and “multi-shelled” are thus used synonymously.

“Stability layer” refers to the layer of a capsule wall that is essentially responsible for the stability of the capsule shell, i.e., usually constitutes the main part of the shell.

“Wall formers” are the components that build up the microcapsule wall.

“Hydrogenated castor oil” refers to partially or fully hydrogenated castor oil (castor oil, hydrogenated). Castor oil (CAS No. 8001-79-4) is a well-known vegetable oil, which consists of 80-85% of the triglyceride of ricinoleic acid (triicinolein). Other components include glycerides of various other fatty acids and a small amount of free fatty acids. Through hydrogenation, triricinolein is converted into the triglyceride of 12-hydroxystearic acid. According to the invention, ethoxylated, hydrogenated castor oils are used, which are usually obtained by reacting hydrogenated castor oil with ethylene oxide. The compounds thus obtained and used in the invention contain on average 20-60 ethylene units, with 30 to 50 EO being particularly preferred, and 40 EO being especially preferred. PEG-40 hydrogenated castor oil (INCI) is commercially available as Eumulgin® HRE 40 from BASF, for example. Such compounds are suitable as nonionic oil-in-water emulsifiers and are offered and used as such.

### Microcapsules

The biodegradable microcapsules comprise a core material and a shell, wherein the shell consists of at least one barrier layer and one stability layer, wherein the barrier layer surrounds the core material, wherein the stability layer comprises at least one biopolymer and is arranged on the outer surface of the barrier layer, and wherein preferably an emulsion stabilizer is arranged at the transition from the barrier layer to the stability layer. This arrangement can consist of an intermediate layer of emulsion stabilizer, which can be continuous or discontinuous and can cover parts or the entire inner barrier layer. Alternatively, individual molecules of the emulsion stabilizer can be arranged on the surface of the barrier layer in such a way that they mediate a bond between the stability layer and the barrier layer. The emulsion stabilizer acts here as a linking agent.

The microcapsule shells exhibit a significantly increased thickness of the stability layer due to the application of the emulsion stabilizer. As a result, the proportion of natural components in the capsule is further increased compared to previously described multilayer microcapsules.

According to one embodiment of the biodegradable microcapsules, during the production of the microcapsules, the surface of the barrier layer is brought into contact with the emulsion stabilizer before the formation of the stability layer. This increases the capacity of the surface for structural attachment of the stability layer. Without wanting to be bound by theory, the inventors assume that the emulsion stabilizer attaches to the partially polar surface of the barrier layer, in particular a melamine-resorcin-formaldehyde layer, thus providing both a framework to the biopolymers of the stability layer and the sufficient non-polar surface characteristics for the deposition of the formed coacervate. This not only increases the average layer thickness of the stability layer produced with the biopolymer but also incorporates the emulsion stabilizer at the interface between the stability layer and the barrier layer. Based on this theory, essentially any emulsion stabilizer could act as a linking agent for the production of the microcapsules according to the invention.

In a preferred embodiment, the emulsion stabilizer is a polymer or copolymer, consisting of one or more monomers selected from:
(1) Acrylic acid derivatives of the general formula (I)

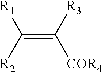

wherein
R₁, R₂ and Ra are selected from hydrogen and an alkyl group with 1 to 4 carbon atoms, wherein R₁ and R₂ are particularly hydrogen and R₃ is particularly hydrogen or methyl; and
R₄ is for —OX or —NR₅R₆, wherein X is hydrogen, an alkali metal, an ammonium group, or a C₁-C₁₀ alkyl possibly substituted by —SO₃M or —OH, wherein M is hydrogen, an alkali metal, or ammonium, wherein the optionally —SO₃M or —OH substituted C₁-C₁₈ alkyl is preferably methyl, ethyl, n-butyl, 2-ethylhexyl, 2-sulfoethyl, or 2-sulfopropyl, wherein R₅ and R₆ are independently selected from hydrogen or a C₁-C₁₀ alkyl possibly substituted by —SO₃M, wherein at least one of R₅ and R₆ is not hydrogen, and wherein preferably R₅ is H and R₆ is 2-methyl-propan-2-yl-1-sulfonic acid;
(2)N-vinylpyrrolidone; and
(3) styrene.

The possible C₁₋₄ hydroxyalkyl groups for R₁, R₂, and R₃ can be ethyl, n-propyl, i-propyl, and n-butyl. In some embodiments of the acrylic acid derivatives, R₁ and R₂ are hydrogen, and R₃ is hydrogen or methyl. Depending on the selection of R₃, it is an acrylate (hydrogen) or methacrylate (methyl).

The possible C₁-C₁₈ alkyl groups for X, which may be substituted by —OH or —SO₃M, are preferably selected from methyl, ethyl, C₂₋₄ hydroxyalkyl, C₂₋₄ sulfoalkyl, and C₄-C₁₈ alkyl groups.

The C₂₋₄ hydroxyalkyl groups can be selected from ethyl, n-propyl, i-propyl, and n-butyl. As unsubstituted C₄-18alkyl groups, examples include n-butyl, sec-butyl, tert-butyl, pentyl, hexyl, ethylhexyl, octyl, decyl, dodecyl, or stearyl groups. Particularly suitable are n-butyl and ethylhexyl. Ethylhexyl is particularly 2-ethylhexyl. As C₂₋₄ sulfoalkyl groups, particularly 2-sulfoethyl and 3-sulfopropyl are noteworthy.

According to one embodiment of the acrylic acid derivatives, R₄ is —NR₅R₆, where R₅ is H and R₆ is 2-methylpropan-2-yl-1-sulfonic acid. Particularly in this case, R₁, R₂, and R₃ are hydrogen.

According to one embodiment, R₄ is —OX and X is hydrogen. Particularly, R₁, R₂, and R₃ are hydrogen (acrylic acid). Alternatively, R₃ is methyl (methacrylate). According to an embodiment of the acrylic acid derivatives, R₄ is —OX and X is methyl. Particularly, R₁, R₂, and R₃ are hydrogen (methyl acrylate). According to one embodiment, R₄ is —OX and X is 2-ethylhexyl. Particularly, R₁, R₂, and R₃ are hydrogen (ethylhexyl acrylate). According to one embodiment, R₄ is —OX and X is n-butyl. Particularly, R₁, R₂, and R₃ are hydrogen (n-butyl acrylate). According to one embodiment of the acrylic acid derivatives, R₄ is —OX and X is 2-sulfoethyl. Particularly, R₁, R₃, and R₃ are hydrogen (sulfoethyl acrylate). According to one embodiment of the acrylic acid derivatives, R₄ is —OX and X is 3-sulfopropyl. Particularly, R₁, and R₂ are hydrogen and R₃ is methyl (sulfopropyl(meth)acrylate).

The polymers or copolymers composed of monomers of formula (I) typically conform to formula (II):

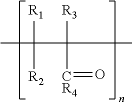

wherein R₁-R₄ have the meanings given above and n is a whole number of at least 3. For example, n may be greater than 5, 10, 20, 30, 40, 50, 60, 70, 80, or 100, n may be, for example, less than 10,000, 7,500, 5,000, 2,500, 1,000, or 500. According to one embodiment, n is in the range of 5 to 5,000. In one embodiment, n is in the range of 10 to 1,000

This group of polymers and copolymers represents a sensible generalization of the copolymers present in the commercially available emulsion stabilizer “Dimension PA 140”. The emulsion stabilizer is preferably an acrylate copolymer containing at least two different monomers of formula (I). In various embodiments, the copolymer contains AMPS, optionally in combination with (meth)acrylic acid and/or at least one alkyl (meth)acrylate. According to one embodiment, the copolymer contains AMPS and one or more monomers selected from acrylate, methacrylate, methyl acrylate, ethylhexyl acrylate, n-butyl acrylate, N-vinylpyrrolidone, and styrene.

According to one embodiment, the copolymer contains AMPS, acrylate, methyl acrylate, and styrene. According to another embodiment, the copolymer contains AMPS, acrylate, methyl acrylate, and ethylhexyl acrylate. According to one embodiment, the copolymer contains AMPS, methyl acrylate, N-vinylpyrrolidone, and styrene. According to one embodiment, the copolymer contains AMPS, acrylate, methyl acrylate, and ethylhexyl acrylate. According to one embodiment, the copolymer contains AMPS, methyl acrylate, N-vinylpyrrolidone, and styrene. According to one embodiment, the copolymer contains AMPS, methyl acrylate, and styrene. According to one embodiment, the copolymer contains AMPS, methacrylate, and styrene. According to one embodiment, the copolymer contains AMPS, acrylate, methyl acrylate, and n-butyl acrylate.

According to one embodiment, the emulsion stabilizer is a copolymer as defined in EP0562344B1, which is incorporated herein by reference. According to one embodiment, the emulsion stabilizer is a copolymer containing a) AMPS, sulfoethyl or sulfopropyl (meth)acrylate, or vinyl sulfonic acid, particularly with a proportion of 20 to 90%; b) a vinyl unsaturated acid, particularly with a proportion of 0 to 50%; c) methyl or ethyl acrylate or methacrylate, C₂₋₄ hydroxyalkyl acrylate, or N-vinylpyrrolidone, particularly with a proportion of 0 to 70%; and d) styrene or C₄₋₁₈ alkyl acrylate or C₄₋₁₈ alkyl methacrylate, particularly with a proportion of 0.1 to 10%.

According to one embodiment, the emulsion stabilizer is a copolymer containing: a) 2-acrylamido-2-methylpropanesulfonic acid, sulfoethyl or sulfopropyl(meth)acrylate or vinylsulfonic acid, particularly in an amount of 40 to 75%; b) acrylic acid or methacrylic acid, particularly in an amount of 10 to 40%; c) methyl or ethyl acrylate or methacrylate, C₂₋₄ hydroxyalkyl acrylate, or N-vinylpyrrolidone, particularly in an amount of 10 to 50%; and d) 0.5 to 5% styrene or C₄₋₁₈ alkyl acrylate or methacrylate, particularly in an amount of 0.5 to 5%.

According to one embodiment, the emulsion stabilizer is a copolymer containing: a) 40 to 75% 2-acrylamido-2-methylpropanesulfonic acid, sulfoethyl or sulfopropyl(meth)acrylate, or vinylsulfonic acid, particularly in an amount of 40 to 75%; b) acrylic acid or methacrylic acid, 10 to 30%; c) methyl or ethyl acrylate or methacrylate, or N-vinylpyrrolidone, particularly in an amount of 10 to 50%; and d) styrene or C₄₋₁₈ alkyl acrylate or methacrylate, particularly in an amount of 0.5 to 5%.

A suitable copolymer is commercially available under the trade name Dimension PA 140 (Solenis).

The exact determination of the proportion of the emulsion stabilizer in the stabilization layer is technically challenging. However, unlike its use as a protective colloid in the encapsulation of the core material, it is assumed that a significant part of the emulsion stabilizer is incorporated into the microcapsule shell.

The proportion of the emulsion stabilizer used in the components for microencapsulation can range from 0.1 wt. % to 15 wt. %. For example, the proportion of the emulsion stabilizer used can be 0.1 wt. %, 0.2 wt. %, 0.5 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %. Below a concentration of 0.1 wt. %, there is a risk that the surface of the barrier layer is not sufficiently covered with the emulsion stabilizer to ensure the desired effect of increasing the deposition amount of the stability layer. Above 15 wt. %, the high concentration of an emulsion stabilizer can hinder the formation of the stability layer. In a preferred embodiment, the emulsion stabilizer is used in an amount of 0.25 wt. % to 5 wt. % of the components used for microencapsulation. In a particularly preferred embodiment, the proportion of the emulsion stabilizer used is in the range of 0.5 wt. % to 4 wt. %.

Assuming that at least part of the emulsion stabilizer is incorporated into the microcapsule wall, according to one embodiment, the proportion of the emulsion stabilizer based on the total weight of the microcapsule wall is in the range of 0.5 wt. % to 15.0 wt. %. For example, the proportion of the emulsion stabilizer used can be 0.5 wt. %, 1.0 wt. %, 1.5 wt. %, 2.0 wt. %, 2.5 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %. In a preferred embodiment, the proportion of the wall-forming components of the microcapsule shell is in the range of 1 wt. % to 11 wt. %. In a particularly preferred embodiment, the proportion of the emulsion stabilizer used is in the range of 2 wt. % to 7 wt. %.

The barrier layer preferably contains, as wall-formers, one or more components selected from the group consisting of an aldehyde component, an aromatic alcohol, an amine component, and an acrylate component. Methods for producing microcapsules with these wall materials are known to the skilled person. For the production of the barrier layer, a polymer selected from a polycondensation product of an aldehyde component with one or more aromatic alcohols and/or amine components can be used.

The low wall thickness of the barrier layer can be achieved particularly with a melamine-formaldehyde layer containing aromatic alcohols or m-aminophenol. Accordingly, the barrier layer preferably comprises an aldehyde component, an amine component, and an aromatic alcohol.

The use of amine-aldehyde compounds in the barrier layer, particularly melamine-formaldehyde, has the advantage that these compounds form a hydrophilic surface with a high proportion of hydroxyl functionality, thus exhibiting fundamental compatibility with the hydrogen-bond-oriented components of the first layer (stability layer), such as biodegradable proteins, polysaccharides, chitosan, lignins, and phosphazenes, as well as inorganic wall materials like CaCO3 and polysiloxanes. Similarly, polyacrylates, particularly from the components styrene, vinyl compounds, methyl methacrylate, and 1,4-butanediol acrylate, methacrylic acid, can be produced as a microcapsule wall by initiation, e.g., with t-butyl hydroperoxide in a radically induced polymerization (polyacrylates), forming a hydrophilic surface with a high proportion of hydroxyl functionality, which is therefore also compatible with the components of the stability layer.

In a preferred embodiment, a wall-forming agent of the barrier layer is thus an aldehyde component. According to one embodiment, the aldehyde component of the barrier layer is selected from the group consisting of formaldehyde, glutaraldehyde, succinaldehyde, furfural, and glyoxal. Microcapsules have already been successfully produced with all these aldehydes (see WO 2013 037 575 A1), so it can be assumed that similarly dense capsules can be obtained with them as with formaldehyde.

The proportion of the aldehyde component for wall formation can be in the range of 5 wt. % to 50 wt. % based on the total weight of the barrier layer. For example, the proportion of the aldehyde component can be 5 wt. %, 6 wt. %, 7 wt. %, 8 wt. %, 9 wt. %, 10 wt. %, 15 wt. %, 20 wt. %, 25 wt. %, 30 wt. %, 35 wt. %, 40 wt. %, 45 wt. %, or 50 wt. %. It is assumed that outside these limits, a sufficiently stable and dense thin layer cannot be obtained. Preferably, the concentration of the aldehyde component in the barrier layer is in the range of 10 wt. % to 30 wt. %. Particularly preferably, the concentration of the aldehyde component in the barrier layer is in the range of 15 wt. % to 20 wt. %.

As amine component in the barrier layer, melamine, melamine derivatives, and urea or combinations thereof are particularly suitable. Suitable melamine derivatives are etherified melamine derivatives as well as methylolated melamine derivatives. Melamine in the methylolated form is preferred. The amine components can be used, for example, in the form of alkylated mono- and polymethylol-urea precondensates or partially methylolated mono- and polymethylol-1,3,5-triamino-2,4,6-triazine precondensates, such as Dimension SD® (Solenis). According to one embodiment, the amine component is melamine. According to an alternative embodiment, the amine component is a combination of melamine and urea.

The aldehyde component and the amine component can be present in a molar ratio in the range of 1:5 to 3:1. For example, the molar ratio can be 1:5, 1:4.5, 1:4, 1:3.5, 1:3, 1:2.5, 1:2, 1:1.8, 1:1.6, 1:1.4, 1:1.35, 1:1.3, 1:1.2, 1:1, 1.5:1, 2:1, 2.5:1, or 3:1. Preferably, the molar ratio is in the range of 1:3 to 2:1. Particularly preferably, the molar ratio of the aldehyde component and the amine component is in the range of 1:2 to 1:1. The aldehyde component and the amine component are usually used in a ratio of about 1:1.35. This molar ratio allows for a complete reaction of the two reactants and leads to a high impermeability of the capsules. For example, aldehyde-amine capsule walls with a molar ratio of 1:2 are also known. These capsules have the advantage of a very low proportion of the highly cross-linking aldehyde, particularly formaldehyde.

However, these capsules have lower impermeability than the capsules with a ratio of 1:1.35. Capsules with a ratio of 2:1 have increased impermeability but have the disadvantage that the aldehyde component is partially unreacted in the capsule wall and the slurry.

In one embodiment, the proportion of the amine component(s) (e.g., melamine and/or urea) in the barrier layer, based on the total weight of the barrier layer, is in the range of 20 wt. % to 85 wt. %. For example, the proportion of the amine component can be 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, or 85 wt. %. In a preferred embodiment, the proportion of the amine component in the barrier layer, based on the total weight of the barrier layer, is in the range of 40 wt. % to 80 wt. %. Particularly preferably, the proportion of the amine component is in the range of 55 wt. % to 70 wt. %.

With the aromatic alcohol, it is possible to significantly reduce the wall thickness of the barrier layer constructed from the amine component and the aldehyde component while still obtaining a layer that has the necessary impermeability and is stable enough, at least in combination with the stability layer. The aromatic alcohols provide the wall with increased impermeability because their highly hydrophobic aromatic structure makes it difficult for low molecular weight substances to diffuse through. As demonstrated in the examples, phloroglucinol, resorcinol, or m-aminophenol are particularly suitable as aromatic alcohols. Consequently, in one embodiment, the aromatic alcohol is selected from the group consisting of phloroglucinol, resorcinol, and aminophenol. In combination with the amine and aldehyde components, the aromatic alcohol is used in a molar ratio to the aldehyde component in the range of (alcohol:aldehyde) 1:1 to 1:20, preferably in the range of 1:2 to 1:10.

In one embodiment, the proportion of the aromatic alcohol in the barrier layer, based on the total weight of the barrier layer, is in the range of 1.0 wt. % to 20 wt. %. For example, the proportion of the aromatic alcohol can be 1.5 wt. %, 2.0 wt. %, 2.5 wt. %, 3.0 wt. %, 4.0 wt. %, 5.0 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, or 20 wt. %.

Due to their aromatic structure, the aromatic alcohols impart a coloration to the capsule wall, which increases with the proportion of aromatic alcohol. Such coloration is undesirable in a variety of applications. Moreover, aromatic alcohols are prone to oxidation, which leads to a change in coloration over time. This unwanted coloration of the microcapsules is difficult to balance with a dye. Therefore, aromatic alcohols should not be used above 20.0 wt. %. Below 1.0 wt. %, no effect regarding impermeability is detectable. In a preferred embodiment, the proportion of the aromatic alcohol in the barrier layer, based on the total weight of the barrier layer, is in the range of 5.0 wt. % to 15.0 wt. %. Up to a percentage of 15.0 wt. %, the coloration is tolerable in most applications. In a particularly preferred embodiment, the proportion of the aromatic alcohol in the barrier layer, based on the total weight of the barrier layer, is in the range of 6 wt. % to 16.0 wt. %. In particular, the proportion of the aromatic alcohol in the barrier layer is in the range of 10 wt. % to 14.0 wt. %.

In another embodiment, the aldehyde component of the barrier layer can be used together with an aromatic alcohol such as resorcinol, phloroglucinol, or m-aminophenol as the wall-forming component(s), i.e., without the amine component(s).

In one embodiment, the barrier layer contains melamine, formaldehyde, and resorcinol. In another embodiment, the barrier layer of the microcapsules contains melamine, urea, formaldehyde, and resorcinol. In a preferred embodiment, the barrier layer contains melamine in the range of 25 to 40 wt. %, formaldehyde in the range of 15 to 20 wt. %, and resorcinol in the range of 10 to 14 wt. %, and optionally urea in the range of 25 to 35 wt. %. The proportions refer to the amounts used for the wall formation of the layer and are based on the total weight of the barrier layer without the protective colloid.

For the encapsulation of the core material with the barrier layer consisting of an aldehyde component, an amine component, and an aromatic alcohol, it is preferred to use an emulsion stabilizer as a protective colloid, as mentioned above. The emulsion stabilizer used as a protective colloid can be a polymer or copolymer defined as a mediator agent as described above. For example, the protective colloid can be a copolymer containing AMPS (Dimension® PA 140, Solenis) or its salts. In one embodiment, the same copolymer is used as both the protective colloid and the mediator agent.

As an amine component in the barrier layer, melamine, melamine derivatives, and urea or combinations thereof are particularly suitable. Suitable melamine derivatives are etherified melamine derivatives as well as methylolated melamine derivatives, with melamine in the methylolated form being preferred. The amine components can be used, for example, in the form of alkylated mono- and polymethylol-urea precondensates or partially methylolated mono- and polymethylol-1,3,5-triamino-2,4,6-triazine precondensates, such as Dimension SD® (Solenis). According to one embodiment, the amine component is melamine. According to an alternative embodiment, the amine component is a combination of melamine and urea.

The stability layer forms the main component of the microcapsule shell, thus ensuring high biodegradability according to OECD 301 F of at least 40% within 60 days. Biopolymers suitable as wall-forming agents for the stability layer are proteins such as gelatin, whey protein, plant storage protein; polysaccharides such as alginate, gum arabic, modified gum, chitin, dextran, dextrin, pectin, cellulose, modified cellulose, hemicellulose, starch or modified starch; phenolic macromolecules such as lignin; polyglucosamines such as chitosan, polyvinyl esters such as polyvinyl alcohols and polyvinyl acetate: phosphazenes and polyesters such as polylactide or polyhydroxyalkanoate. This list of specific components within the individual material classes is exemplary and should not be considered limiting. Suitable natural wall-forming agents are known to those skilled in the art. Furthermore, the various methods for wall formation, such as coacervation or interfacial polymerization, are known to the skilled person.

The biopolymers can be selected according to the respective application in order to form a stable multi-layer shell with the material of the stability layer. Furthermore, the biopolymers can be selected to achieve compatibility with the chemical conditions of the application area. The biopolymers can be combined in various ways to influence the biodegradability or, for example, the stability and chemical resistance of the microcapsule.

According to one embodiment of the first aspect, the shell of the microcapsules exhibits a biodegradability of 50% according to OECD 301 F. In another embodiment, the shell of the microcapsule exhibits a biodegradability of at least 60% (OECD 301 F). In another embodiment, the biodegradability is at least 70% (OECD 301 F). The biodegradability is measured over a period of 60 days. In the extended biodegradation process (“enhanced ready biodegradation”), the biodegradability is measured over a period of 60 days (see Opinion on an Annex XV dossier proposing restrictions on intentionally-added microplastics of Jun. 11, 2020, ECHA/RAC/RES-O-0000006790-71-01/F). The microcapsules are preferably freed from residues by washing before determining the biodegradability. Particularly preferred are copies of the microcapsules for this test made with an inert, non-biodegradable core material like perfluorooctane (PFO) instead of perfume oil. In one embodiment, the capsule dispersion is washed after production by threefold centrifugation and redispersion in distilled water. For this, the sample is centrifuged (e.g., for 10 min at 12,000 RPM). After removing the clear supernatant, it is filled with water and the sediment is redispersed by shaking. Various reference samples can be used in the measurement of biodegradability, such as the rapidly degradable ethylene glycol or natural-based walnut shell flour with the typical step-like degradation of a complex mixture of substances. The microcapsule according to the invention shows similar, preferably better, biodegradability over a period of 28 or 60 days than the walnut shell flour.

Residues in the microcapsule dispersions are substances used in the production of the microcapsules and interact non-covalently with the shell, such as deposition aids, preservatives, emulsifiers/protective colloids, and excess ingredients. These residues have a proven impact on the biodegradability of microcapsule dispersions. Therefore, washing before determining the biodegradability is necessary.

To gain an understanding of the proportion of covalently and non-covalently bound components in the microcapsule dispersion, the capsules were analyzed using the quantification method based on Py-GC-MS for polymer-encapsulated fragrances as described in Gasparini et al. 2020 (Gasparini G, Semaoui S, Augugliaro J, Boschung A, Berthier D, Seyfried M, Begnaud F. Quantification of Residual Perfume by Py-GC-MS in Fragrance Encapsulate Polymeric Materials Intended for Biodegradation Tests. Molecules. 2020; 25(3):718). This method involves a multi-step cleaning protocol for polymers from complex samples like microcapsule dispersions and allows the quantification of volatile residual components, which are presumed not to be covalently bound into the 3D polymer network and are therefore not quantifiable with other standard methods (e.g., SPME-GC-MS or TGA). This procedure confirmed that individual layers of the microcapsule according to the invention, particularly the barrier and stability layers, are inseparably connected and can be considered as a monopolymer. It is assumed that the addition of the emulsion stabilizer not only improves the structural integration of the stability layer by the barrier layer but also increases the structural (covalent) connection of all wall-forming components.

A high value of biodegradability is achieved on the one hand through the wall-forming agents used, and on the other hand through the structure of the shell. The use of a certain percentage of biopolymers does not automatically lead to a corresponding value of biodegradability. This depends on how the biopolymers are present in the shell.

According to a preferred embodiment, the stability layer contains gelatin as a biopolymer. According to another preferred embodiment, the stability layer contains alginate as a biopolymer. According to another preferred embodiment, the stability layer contains gelatin and alginate as biopolymers. Both gelatin and alginate are suitable for the production of microcapsules with high biodegradability and high stability. Particularly with a stability layer containing gelatin and alginate, treating the surface of the barrier layer with an emulsion stabilizer, particularly a copolymer containing AMPS, can lead to a significant increase in the layer thickness of the stability layer. Other suitable combinations of natural components in the first layer (stability layer) are gelatin and gum arabic.

The stability layer contains one or more curing agents. Suitable curing agents are aldehydes such as glutaraldehyde, formaldehyde, and glyoxal, as well as tannins, enzymes such as transglutaminase, and organic anhydrides such as maleic anhydride, epoxy compounds, polyvalent metal cations, amines, polyphenols, maleimides, sulfides, phenol oxides, hydrazides, isocyanates, isothiocyanates, N-hydroxysulfosuccinimide derivatives, carbodiimide derivatives, and polyols. The preferred curing agent is glutaraldehyde due to its very good cross-linking properties. Another preferred curing agent is glyoxal due to its good cross-linking properties and, compared to glutaraldehyde, lower toxicological classification. The use of curing agents results in a higher impermeability of the stability layer. However, curing agents lead to a reduced biodegradability of the natural polymers.

Due to the presence of the barrier layer as a diffusion barrier, the amount of curing agent in the stability layer can be kept low, which in turn contributes to the easy biodegradability of the layer. According to one embodiment, the proportion of the curing agent in the stability layer is below 25 wt. %. Unless explicitly defined otherwise, the proportions of the components of the layers refer to the total weight of the layer, i.e., the total dry weight of the components used for production, excluding components used in production that are not or only minimally incorporated into the layer, such as surfactants and protective colloids. Above this value, biodegradability according to OECD 301 F cannot be guaranteed. The proportion of the curing agent in the stability layer can be, for example, 1.0 wt. %, 2.0 wt. %, 3.0 wt. %, 4.0 wt. %, 5.0 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, or 24 wt. %. Preferably, the proportion of the curing agent in the stability layer is in the range of 1 wt. % to 15 wt. %. This proportion leads to the effective cross-linking of the gelatin and results in a quantitative reaction with minimal residual monomer. The range of 9 to 12 wt. % is particularly preferred, as it ensures the required degree of cross-linking and provides a stable coating of the barrier layer to buffer the otherwise sensitive barrier layer, with only a small amount of residual aldehyde, which is degraded in a subsequent alkaline adjustment of the slurry via an aldol reaction.

In one embodiment, the stability layer contains gelatin and glutaraldehyde. In another embodiment, the stability layer contains gelatin, alginate, and glutaraldehyde. In an additional embodiment, the stability layer contains gelatin and glyoxal. In another embodiment, the stability layer contains gelatin, alginate, and glyoxal. The exact chemical composition of the stability layer is not critical. However, the desired effect is preferably achieved with polar biopolymers.

According to one embodiment, the microcapsule shell contains no titanium dioxide. According to another embodiment, the microcapsule shell contains no metal oxide. According to another embodiment, the microcapsule shell contains no pigment. According to another embodiment, the microcapsule shell contains no dye.

By using the emulsion stabilizer on the surface of the barrier layer, the average thickness of the stability layer is significantly increased. The average thickness of the stability layer is at least 1 μm. The average thickness of the stability layer can be 1 μm, 1.2 μm, 1.4 μm, 1.6 μm, 1.8 μm, 2 μm, 2.2 μm, 2.4 μm, 2.6 μm, 2.8 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, or 10 μm. The stability layer often has an elliptical shape in cross-section, causing the thickness of the stability layer to vary across the microcapsule surface. Therefore, an average thickness of the microcapsules is calculated. Additionally, the deposition varies from microcapsule to microcapsule. This is accounted for by determining the average thickness of several microcapsules and calculating the mean. Thus, the average thickness mentioned here is precisely an average mean thickness. The determination of the layer thickness of the stability layer can be carried out in two ways. First, the light microscopic approach is mentioned here, i.e., the direct, optical measurement of the observed layer thickness using a microscope and appropriate software. In this method, a large number of microcapsules in a dispersion are measured, and the minimum diameter of each individual microcapsule is determined due to the variance within the capsules.

A second option is the measurement of the particle size distribution using laser diffraction. Here, the modal value of a particle size distribution without the layer to be measured can be compared to the modal value of a particle size distribution with the layer to be measured. The enlargement of this modal value indicates the increase in the hydrodynamic diameter of the main fraction of the measured microcapsules. Ultimately, the difference between the two measured modal values yields twice the layer thickness of the layer.

According to a preferred embodiment, the average thickness of the stability layer is at least 2 μm. By choosing a suitable combination of emulsion stabilizer and wall-forming agents for the stability layer, stability layers with an average thickness of 6 μm or more can be formed. In a particularly preferred embodiment, the average thickness of the stability layer is at least 3 μm.

In contrast to other biodegradable microcapsules, the microcapsules described herein exhibit high impermeability. According to one embodiment, the microcapsules have an impermeability that ensures a leakage of no more than 50 wt. % of the encapsulated core material after storage for a period of 4 weeks at a temperature of 0 to 40° C.

Besides the shell material, the impermeability also depends on the type of core material. The impermeability of the microcapsules was determined, for example, for the fragrance oil Weiroclean by Kitzing, as this fragrance oil is representative of microencapsulated fragrance oils in its chemical properties. Weiroclean contains the following components (with proportion based on the total weight):

| | |
|---|---|
| 1-(1,2,3,4,5,6,7,8-Octahydro-2,3,8,8-tetramethyl-2- | 25-50% |
| naphthalenyl)ethanone | |
| Benzoic Acid, 2-hydroxy-, 2-hexyl ester | 10-25% |
| Phenylmethyl benzoate | 5-10% |
| 3-Methyl-4-(2,6,6-trimethyl-2-cyclohexenyl)-3-buten-2-one | 1-5% |
| 3,7-Dimethyl-6-octen-1-ol | 1-5% |
| 3-Methyl-5-phenylpentanol | 1-5% |
| 2,6-Dimethyloct-7-en-2-ol | 1-5% |
| 4-(2,6,6-Trimethylcyclohex-1-eneyl)-but-3-ene-2-one | 1-5% |
| 3a,4,5,6,7,7a-Hexahydro-4,7-methano-1H-inden-6-yl | 1-5% |
| Propanoate | |
| 2-tert-Butylcyclohexyl acetate | 1-5% |
| 2-Heptylcyclopentanone | 1-5% |
| Pentadecan-15-olide | 1-5% |
| 2H-1-Benzopyran-2-one | 0.1-1% |
| 2,6-Di-tert-butyl-p-cresol | 0.1-1% |
| 4-Methyl-3-decen-5-ol | 0.1-1% |
| 2,4-Dimethyl-3-cyclohexen-1-carboxaldehyde | 0.1-1% |
| [(2E)-3,7-dimethylocta-2,6-dienyl] acetate | 0.1-1% |
| Allyl hexanoate | 0.1-1% |
| 2-Methylundecanal | 0.1-1% |
| 10-Undecenal | 0.1-1% |
| cis-3,7-Dimethyl-2,6-octadienyl ethanoate | 0.1-1% |
| 3,7,11-Trimethyldodeca-1,6,10-trien-3-ol | 0.1-1% |
| Undecan-2-one | 0.1-1% |

A variety of different materials can be used as core material, including fragrances and cosmetic active ingredients. According to a preferred embodiment of the microcapsules, the core material is hydrophobic. The core material can be solid or liquid. In particular, it is liquid. Preferably, it is a liquid hydrophobic core material. In a preferred embodiment, the core material is a fragrance or comprises at least one fragrance. Particularly preferably, it is a fragrance or perfume oil optimized for microencapsulation for the detergent and cleaning agent sector, such as the fragrance formulation Weiroclean (Kurt Kitzing GmbH). The fragrances can be used in the form of a solid or liquid formulation, but particularly in liquid form.

Fragrances that can be used as core material are not subject to any particular restrictions. Individual fragrance compounds of natural or synthetic origin, such as esters, ethers, aldehydes, ketones, alcohols, and hydrocarbons, can be used. Fragrance compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenylglycinate, allylcyclohexylpropionate, styrallyl propionate, benzyl salicylate, cyclohexyl salicylate, Floramat, Melusat, and Jasmacyclat. Examples of ethers include benzylethyl ether and ambroxan; aldehydes include the above-mentioned linear alkanals with 8 to 18 carbon atoms, citral, citronellal, citronellyloxyacetaldehyde, cyclamen aldehyde (3-(4-isopropylphenyl)butanal), lilial, and bourgeonal; ketones include ionones, alpha-isomethyl ionone, and methyl cedryl ketone; alcohols include anethole, citronellol, eugenol, geraniol, linalool, phenylethyl alcohol, and terpineol; hydrocarbons mainly include terpenes such as limonene and pinene. However, mixtures of different fragrances are preferably used to create a pleasant scent.

Suitable fragrance aldehydes can be selected from Adoxal (2,6,10-trimethyl-9-undecenal), anisaldehyde (4-methoxybenzaldehyde), Cymal or cyclamen aldehyde (3-(4-isopropylphenyl)-2-methylpropanal), Nympheal (3-(4-isobutyl-2-methylphenyl)propanal), ethylvanillin, Florhydral (3-(3-isopropylphenyl)butanal), Trifemal (3-phenylbutyraldehyde), Helional (3-(3,4-methylenedioxyphenyl)-2-methylpropanal), heliotropin, hydroxycitronellal, lauraldehyde, Lyral (3- and 4-(4-hydroxy-4-methylpentyl)-3-cyclohexene-1-carboxaldehyde), methylnonylacetaldehyde, Lilial (3-(4-tert-butylphenyl)-2-methylpropanal), phenylacetaldehyde, undecylenaldehyde, vanillin, 2,6,10-trimethyl-9-undecenal, 3-dodecen-1-al, alpha-n-amyl cinnamaldehyde, Melonal (2,6-dimethyl-5-heptenal), Triplal (2,4-dimethyl-3-cyclohexene-1-carboxaldehyde), 4-methoxybenzaldehyde, benzaldehyde, 3-(4-tert-butylphenyl)propanal, 2-methyl-3-(para-methoxyphenyl)propanal, 2-methyl-4-(2,6,6-trimethyl-2(1)-cyclohexene-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzylaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decyliden-8)-butanal, octahydro-4,7-methano-1H-indene carboxaldehyde, 3-ethoxy-4-hydroxybenzaldehyde, para-ethyl-alpha,alpha-dimethylhydrocinnamaldehyde, alpha-methyl-3,4-(methylenedioxy)hydrocinnamaldehyde, 3,4-methylenedioxybenzaldehyde, alpha-n-hexyl cinnamaldehyde, m-cymen-7-carboxaldehyde, alpha-methylphenylacetaldehyde, tetrahydrocitral (3,7-dimethyloctanal), undecenal, 2,4,6-trimethyl-3-cyclohexene-1-carboxaldehyde, 4-(3)(4-methyl-3-pentenyl)-3-cyclohexene carboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanoindan-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexene carboxaldehyde, 7-hydroxy-3,7-dimethyloctanal, trans-4-decenal, 2,6-nonadienal, para-tolylacetaldehyde, 4-methylphenylacetaldehyde, 2-methyl-4-(2,6,6-timethyl-1-cyclohexene-1-yl)-2-butenal, ortho-methoxycinnamaldehyde, 3,5,6-trmethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyloctanal, alpha-methyl-4-(1-methylethyl)benzeneacetaldehyde, 6,6-dimethyl-2-norpinene-2-propionaldehyde, para-methylphenoxyacetaldehyde, 2-methyl-3-phenyl-2-propen-1-al, 3,5,5-trimethylhexanal, hexahydro-8,8-dimethyl-2-naphthaldehyde, 3-propylbicyclo[2.2.1]hept-5-en-2-carbaldehyde, 9-decenal, 3-methyl-5-phenyl-1-pentanal, floral (4,8-dimethyl-4,9-decadienal), aldehyde C12MNA (2-methylundecanal), liminal (beta-4-dimethylcyclohex-3-ene-1-propanal), methylnonylacetaldehyde, hexanal, trans-2-hexenal and mixtures thereof.

Suitable fragrance ketones include, but are not limited to, methyl-beta-naphthyl ketone, musk indanone (1,2,3,5,6,7-hexahydro-1,1,2,3,3-pentamethyl-4H-inden-4-one), Calone (methylbenzodioxepinone), Tonalid (6-acetyl-1,1,2,4,4,7-hexamethyltetralin), alpha-damascone, beta-damascone, delta-damascone, iso-damascone, damascenone, methyldihydrojasmonate (Hedione), menthone, carvone, camphor, koavone (3,4,5,6,6-pentamethylhept-3-en-2-one), fenchone, alpha-ionone, beta-ionone, dihydro-beta-ionone, gamma-methyl-ionone, fleuramone (2-heptylcyclopentanone), raspberry ketone methyl ether (4-(4-methoxyphenyl)butan-2-one), dihydrojasmon, cis-jasmon, 1-(1,2,3,4,5,6,7,8-octahydro-2,3,8,8-tetramethyl-2-naphthalenyl)-ethan-1-one and its isomers, methyl cedrenyl ketone, acetophenone, methyl acetophenone, para-methoxyacetophenone, methyl-beta-naphthyl ketone, benzyl acetone, benzophenone, para-hydroxyphenyl butanone, celery ketone (3-methyl-5-propyl-2-cyclohexenone), 6-isopropyldecahydro-2-naphthone, dimethyloctenone, frescomenthe (2-butan-2-ylcyclohexan-1-one), 4-(1-ethoxyvinyl)-3,3,5,5-tetramethylcyclohexanone, methylheptenone, 2-(2-(4-methyl-3-cyclohexen-1-yl)propyl)cyclopentanone, 1-(p-menth-6(2)-yl)-1-propanone, 4-(4-hydroxy-3-methoxyphenyl)-2-butanone, 2-acetyl-3,3-dimethylnorbomane, 6,7-dihydro-1,1,2,3,3-pentamethyl-4(5H)-indalone, 4-damascol, dulcinyl (4-(1,3-benzodioxol-5-yl)butan-2-one), hexalone (1-(2,6,6-trimethyl-2-cyclohexen-1-yl)-1,6-heptadien-3-one), isocyclemon E (2-acetonaphthone-1,2,3,4,5,6,7,8-octahydro-2,3,8,8-tetramethyl), methylnonyl ketone, methylcyclocitron, methyl lavender ketone, orivone (4-tert-amylcyclohexanone), 4-tert-butylcyclohexanone, delphone (2-pentylcyclopentanone), muscone (CAS 541-91-3), neobutenone (1-(5,5-dimethyl-1-cyclohexenyl)pent-4-en-1-one), plicaton (CAS 41724-19-0), veloutone (2,2,5-trimethyl-5-pentylcyclopentan-1-one), 2,4,4,7-tetramethyl-oct-6-en-3-one, tetrameran (6,10-dimethylundecen-2-one) and mixtures thereof.

The core materials can also contain natural fragrance mixtures accessible from plant sources, such as pine, citrus, jasmine, patchouli, rose, or ylang-ylang oil. Also suitable are clary sage oil, chamomile oil, clove oil, lemon balm oil, mint oil, cinnamon leaf oil, linden blossom oil, juniper berry oil, vetiver oil, frankincense oil, Galbanum oil, and labdanum oil, as well as orange blossom oil, neroli oil, orange peel oil, and sandalwood oil. Other conventional fragrances that can be included in the inventive formulations of the present invention are, for example, essential oils such as Angelica root oil, anise oil, amica flower oil, basil oil, bay oil, champaca flower oil, fir needle oil, fir cone oil, elemi oil, Eucalyptus oil, fennel oil, spruce needle oil, Galbanum oil, geranium oil, gingergrass oil, guaiacwood oil, gurjun balsam oil, helichrysum oil, ho oil, ginger oil, iris oil, cajeput oil, calamus oil, chamomile oil, camphor oil, Cananga oil, cardamom oil, Cassia oil, pine needle oil, copaiba balsam oil, coriander oil, spearmint oil, caraway oil, cumin oil, lavender oil, lemongrass oil, lime oil, mandarin oil, lemon balm oil, musk seed oil, myrrh oil, clove oil, neroli oil, niaouli oil, frankincense oil, oregano oil, palmarosa oil, patchouli oil, Peru balsam oil, petitgrain oil, pepper oil, peppermint oil, allspice oil, pine oil, rose oil, rosemary oil, sandalwood oil, celery oil, spike oil, star anise oil, turpentine oil, Thuja oil, thyme oil, Verbena oil, vetiver oil, juniper berry oil, wormwood oil, wintergreen oil, ylang-ylang oil, hyssop oil, cinnamon oil, cinnamon leaf oil, citronella oil, lemon oil, as well as cypress oil, and ambrettolide, ambroxan, alpha-amylcinnamaldehyde, anethole, anisaldehyde, anisalcohol, anisol, methyl anthranilate, acetophenone, benzyl acetone, benzaldehyde, ethyl benzoate, benzophenone, benzyl alcohol, benzyl acetate, benzyl benzoate, benzyl formate, benzyl valerate, bomeol, bomyl acetate, boisambrene forte, alpha-bromostyrene, n-decyl aldehyde, n-dodecyl aldehyde, eugenol, eugenol methyl ether, eucalyptol, famesol, fenchone, fenchyl acetate, geranyl acetate, geranyl formate, heliotropin, heptanoic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmon, camphor, carvacrol, carvone, p-cresol methyl ether, coumarin, p-methoxyacetophenone, methyl-n-amyl ketone, methyl anthranilate, p-methylacetophenone, methylchavicol, p-methylquinoline, methyl-beta-naphthyl ketone, methyl-n-nonylacetaldehyde, methyl-n-nonyl ketone, muscone, beta-naphthol ethyl ether, beta-naphthol methyl ether, nerol, n-nonyl aldehyde, nonyl alcohol, n-octyl aldehyde, p-oxyacetophenone, pentadecanolide, beta-phenylethyl alcohol, phenylacetic acid, pulegone, safrole, isoamyl salicylate, methyl salicylate, hexyl salicylate, cyclohexyl salicylate, santalol, sandalice, skatole, terpineol, thymene, thymol, troenan, gamma-undelactone, vanillin, veratrum aldehyde, cinnamaldehyde, cinnamyl alcohol, cinnamic acid, ethyl cinnamate, benzyl cinnamate, diphenyl oxide, limonene, linalool, linalyl acetate and propionate, melusat, menthol, menthone, methyl-n-heptenone, pinene, phenylacetaldehyde, terpinyl acetate, citral, citronellal and mixtures thereof.

According to one embodiment, the core material does not contain any monosaccharide polyol, particularly no mannitol, erythritol, xylitol, sorbitol, or their mixtures.

The impermeability of the capsule wall can be influenced by the choice of shell components. According to one embodiment, the microcapsules have an impermeability that ensures a leakage of no more than 45 wt. %, no more than 40 wt. %, no more than 35 wt. %, no more than 30 wt. %, no more than 25 wt. %, no more than 20 wt. % of the encapsulated core material after storage for a period of 4 weeks at a temperature of 0 to 40° C. The microcapsules are stored in a model formulation corresponding to the target application. Furthermore, the microcapsules are storage-stable in the product in which they are used, for example, in detergents, fabric softeners, or cosmetic products. The standard formulations of these products are known to those skilled in the art. Typically, the pH value in the vicinity of the microcapsules during storage ranges from 2 to 12.

The microcapsule shells have at least two layers, i.e., they can be, for example, two-layered, three-layered, four-layered, or five-layered. Preferably, the microcapsules are two- or three-layered.

According to one embodiment, the microcapsule has a third layer arranged on the outside of the stability layer. This third layer can be used to adjust the surface properties of the microcapsule for a specific application. Examples include improving the adhesion of the microcapsules to various surfaces and reducing agglomeration. The third layer also binds residual amounts of aldehyde, thus reducing the content of free aldehydes in the capsule dispersion. Furthermore, it can provide additional (mechanical) stability or further increase the impermeability. Depending on the application, the third layer can contain a component selected from amines, organic salts, inorganic salts, alcohols, ethers, polyphosphazenes, and noble metals.

Noble metals increase the impermeability of the capsules and can provide additional catalytic properties to the microcapsule surface or the antibacterial effect of a silver layer. Organic salts, particularly ammonium salts, lead to cationization of the microcapsule surface, which improves adhesion to, for example, textiles. Alcohols also enhance adhesion to substrates through the formation of H-bonds via free hydroxyl groups. An additional polyphosphazene layer or coating with inorganic salts, e.g., silicates, further increases impermeability without affecting biodegradability. According to a preferred embodiment, the third layer contains activated melamine. The melamine captures possible free aldehyde components of the stability and/or barrier layer, increases the impermeability and stability of the capsule, and can also influence the surface properties of the microcapsules, thereby affecting adhesion and agglomeration behavior.

Due to the low wall thicknesses, the proportion of the barrier layer in the shell, based on the total weight of the shell, is at most 30 wt. %. The proportion of the barrier layer in the shell, based on the total weight of the shell, can be, for example, 30 wt. %, 28 wt. %, 25 wt. %, 23 wt. %, 20 wt. %, 18 wt. %, 15 wt. %, 13 wt. %, 10 wt. %, 8 wt. %, or 5 wt. %. For high biodegradability, the proportion is at most 25 wt. % based on the total weight of the shell. Particularly preferably, the proportion of the barrier layer is at most 20 wt. %. The proportion of the stability layer in the shell, based on the total weight of the shell, is at least 40 wt. %. The proportion of the stability layer in the shell, based on the total weight of the shell, can be, for example, 40 wt. %, 43 wt. %, 45 wt. %, 48 wt. %, 50 wt. %, 53 wt. %, 55 wt. %, 58 wt. %, 60 wt. %, 63 wt. %, 65 wt. %, 68 wt. %, 70 wt. %, 75 wt. %, 80 wt. %, 85 wt. %, or 90 wt. %. For high biodegradability, the proportion of the stability layer is at least 50 wt. %, particularly preferably at least 60 wt. %. The proportion of the third layer in the shell, based on the total weight of the shell, is at most 35 wt. %. The proportion of the third layer in the shell, based on the total weight of the shell, can be, for example, 35 wt. %, 33 wt. %, 30 wt. %, 28 wt. %, 25 wt. %, 23 wt. %, 20 wt. %, 18 wt. %, 15 wt. %, 13 wt. %, 10 wt. %, 8 wt. %, or 5 wt. %. For high biodegradability, the proportion of the third layer is preferably at most 30 wt. %, particularly preferably at most 25 wt. %.

The size of the microcapsules is within the usual range for microcapsules. The diameter can range from 100 nm to 1 mm. The diameter depends on the exact capsule composition and the manufacturing process. The peak maximum of the particle size distribution is regularly used as a measure for the size of the capsules. Preferably, the peak maximum of the particle size distribution is in the range of 1 μm to 500 μm. The peak maximum of the particle size distribution can be, for example, at 1 μm, 2 μm, 3 μm, 4 μm, 5 μm, 10 μm, 15 μm, 20 μm, 30 μm, 40 μm, 50 μm, 60 μm, 70 μm, 80 μm, 90 μm, 100 μm, 120 μm, 140 μm, 160 μm, 180 μm, 200 μm, 250 μm, 300 μm, 350 μm, 400 μm, 450 μm, or 500 μm. According to a particularly preferred embodiment, the microcapsules have a peak maximum of the particle size distribution from 10 μm to 100 μm. In particular, the peak maximum of the particle size distribution is in the range of 10 μm to 50 μm.

The use of the emulsion stabilizer for coating the barrier layer represents a new application, distinct from the usual use of the emulsion stabilizer, namely stabilizing the core material droplets.

### Microcapsule Slurry

The microcapsules described herein are typically pre-formulated in the form of a suspension, also referred to as a slurry. For this purpose, the capsules are dispersed in an aqueous medium to create a suspension of the capsules in the liquid medium.

In the context of the present invention, the term “slurry” refers to a typically aqueous suspension of the perfume microcapsules as defined above. The liquid medium (continuous phase) preferably consists predominantly, i.e., more than 50 wt. %, of water, for example, more than 60 wt. %, more than 70 wt. %, or more than 80 wt. %, but can also consist almost entirely or entirely, i.e., 90 wt. %, 95 wt. % or more, of water. The slurry is preferably pourable, i.e., it can be poured out of a container by tilting the container. A pourable slurry is particularly understood to be a capsule-liquid mixture that has a viscosity below 104 mPas, preferably below 103 mPas, at the processing temperature, preferably at a maximum of 40° C., particularly at a maximum of 20° C. (Brookfield rotational viscometer; spindle 2, 20 rpm).

In addition to the emulsifier used according to the invention, the slurry may contain further auxiliaries, for example, those that ensure a certain shelf life or stability. Commonly used auxiliaries include, for example, surfactants, particularly anionic and/or nonionic surfactants, which are different from the emulsifier used according to the invention.

As described above, emulsifiers/surfactants from the class of ethoxylated, hydrogenated castor oils are used as additives in the slurries of the microcapsules described herein (INCI: ethoxylated hydrogenated castor oil), particularly those with 20 to 60, 30 to 50, or about 40 EO. The latter are also known as PEG-40 hydrogenated castor oil and are commercially available, for example, as Eumulgin® HRE 40 from BASF.

These emulsifiers are used in the slurries in amounts of up to 50 wt. %, preferably up to 40 wt. %, up to 30 wt. % or up to 20 wt. %, particularly preferably with a maximum of 10 wt. %, with typical amounts ranging from at least 0.5 wt. %, at least 1 wt. % or at least 2 wt. %, particularly in ranges of 2 wt. % to 10 wt. %, 3 wt. % to 9 wt. % or 4 wt. % to 8 wt. % or approximately 4 wt. %, 5 wt. %, 6 wt. % or 7 wt. %. When the emulsifier is a component of the continuous phase, this phase preferably contains more than 50 wt. % water and the sum of water and emulsifier preferably constitutes at least 70 wt. %, more preferably at least 80 wt. %, and even more preferably at least 90 wt. % of the continuous phase.

In various embodiments, the continuous phase consists of water and at least one emulsifier.

In various embodiments, the continuous phase contains 60 wt. % to 95 wt. %, preferably 70 wt. % to 95 wt. % water and 2 wt. % to 40 wt. %, preferably 2 wt. % to 20 wt. % of at least one emulsifier.

It has surprisingly been found that these emulsifiers can stabilize the slurries, whereas other common emulsifiers, such as hydroxypropyl guar (CAS 39421-75-5, e.g., Jaguar HP105), ethoxylated C₁₂₋₁₈ fatty alcohols (such as Dehydol® LT5), and ethoxylated sorbitan monosterates, such as polyoxyethylene sorbitan monopalmitate (CAS 9005-66-7), could not achieve sufficient stabilization.

Although the use of emulsifiers other than those according to the invention is possible, it is not preferred according to the invention. In particular, the microcapsule dispersion does not contain potassium cetyl phosphate. However, in addition to the emulsifiers according to the invention, other auxiliaries and additives that are not emulsifiers or surfactants can be used. According to one embodiment, the composition does not contain hydroxylated diphenylmethane derivatives.

In various embodiments, the previously described capsules are present in an amount of 1 wt. % to 60 wt. % based on the total weight of the microcapsule dispersion. The microcapsules can be present, for example, in an amount of 2 wt. %, 4 wt. %, 6 wt. %, 8 wt. %, 10 wt. %, 12 wL.%, 14 wt. %, 16 wt. %, 18 wt. %, 20 wt. %, 22 wt. %, 24 wt. %, 26 wt. %, 28 wt. %, 30 wt. %, 32 wt. %, 34 wL %, 36 wt. %, 38 wt. %, 40 wt. %, 42 wt. %, 44 wt. %, 46 wt. %, 48 wt. %, 50 wt. %, 52 wt. %, 54 wt. %, 56 wt. %, 58 wt. %, or 60 wL %. According to one embodiment, the proportion of microcapsules is in the range of 15 wt. % to 50 wL %. According to another embodiment, the proportion of microcapsules is in the range of 20 wt. % to 35 wt. % in the slurry. These dispersions are stable, meaning that even after prolonged storage periods of several days to weeks at typical temperatures up to 40° C., for example, 4 weeks at a temperature between >0 and 40° C., there is no agglomeration, sedimentation, and/or flotation of the capsules or any other phase separation, with this pronounced phase stability attributable to the use of the particular emulsifier.

When producing these slurries, the microcapsules are typically dispersed into an aqueous continuous phase that already contains the emulsifier used according to the invention, using suitable means.

The phase-stabilizing effect occurs over a wide pH range. In particular, the phase-stabilizing effect of the emulsifier is effective at a non-strongly basic pH value. According to one embodiment, the pH of the microcapsule dispersion after adding the emulsifier is less than 11. For example, the pH value of the microcapsule dispersion can be 10.8, 10.5, 10.3, 10.0, 9.8, 9.5, 9.3, 9.0, 8.8, 8.5, 8.3, 8.0, 7.8, 7.5, 7.3, 7.0, 6.8, 6.5, 6.3, or 6.0. The microcapsule dispersion is generally basic. The pH value of the microcapsule dispersion can be less than 10.8, preferably at most 10.5. In one embodiment, the pH value of the microcapsule dispersion is at least 6, preferably at least 7, and particularly preferably at least 8.

The phase-stabilizing effect occurs over a wide conductivity range. The conductivity of the microcapsule dispersion can be at least 6.0 mS/cm. For example, the conductivity can be 6.0 mS/cm, 6.5 mS/cm, 7.0 mS/cm, 7.5 mS/cm, 8.0 mS/cm, 8.5 mS/cm, 9.0 mS/cm, or 10 mS/cm, 10.5 mS/cm, 11 mS/cm, 11.5 mS/cm, 12 mS/cm, 12.5 mS/cm, 13.0 mS/cm, 13.5 mS/cm, 14 mS/cm, 14.5 mS/cm, 15.0 mS/cm. According to one embodiment, the conductivity of the microcapsule dispersion is in the range of 6.0 mS/cm to 15.0 mS/cm, preferably in the range of 8 mS/cm to 12 mS/cm, particularly preferably in the range of 9 mS/cm to 11 mS/cm.

For measuring the pH value and the electrical conductivity of the product or the microcapsule dispersion, the pH/Cond 3320 combination device from WTW can be used. This device is equipped with a pH electrode model “Inlab Expert” (Order Number-5343103) from Mettler Toledo and a conductivity electrode model “Tetra Con 325” from WTW. The glass membrane of the pH electrode is stored in a 3M KCl solution when not in use. Regular calibration of the two electrodes ensures a measurement uncertainty of approximately +/−0.01 pH and +/−0.05 mS/cm. Both the pH and conductivity electrodes are equipped with a temperature sensor, allowing for temperature compensation of the measured values.

To measure the pH value, the pH electrode can be removed from the corresponding 3M KCl storage solution and cleaned with tap water. The electrode is then immersed in the corresponding microcapsule dispersion, ensuring that the entire glass membrane of the electrode is submerged. After stabilizing the reading, the measurement value is read after approximately 5 minutes. The displayed measurement value is dimensionless. Measurements are carried out in undiluted microcapsule dispersions. To measure the electrical conductivity, the cleaned conductivity electrode is immersed in the corresponding microcapsule dispersion. It is ensured that the actual measuring gap of the electrode is fully submerged. After stabilizing the reading, the temperature-compensated measurement value is read after approximately 5 minutes. The displayed measurement value has the unit mS/cm.

### Dried Microcapsule Composition

In another embodiment, the microcapsule dispersion containing the emulsifier can also be in the form of a dried composition, i.e., a powder mixture. The dried microcapsule composition can be obtained in particular by drying a previously described (liquid) microcapsule dispersion. Various methods for drying the liquid microcapsule dispersion are known to the skilled person, including spray drying, fluid bed drying, spray granulation, spray agglomeration, or evaporation.

After drying, the water content in the dried microcapsule dispersion is less than 5 wt. %. The water content can be 5 wt. %, 1 wt. %, 0.8 wt. %, 0.5 wt. %, 0.1 wt. %, 0.05 wt. %, 0.01 wt. %, 0.001 wt. %, or 0.0001 wt. %. According to one embodiment of the dried microcapsule dispersion, the water content is less than 1 wt. %, preferably less than 0.01 wt. %, and even more preferably less than 0.001 wt. %. Particularly preferably, the dried microcapsule dispersion contains no water except for unavoidable traces.

Drying and spray aids can be added to the liquid mixture, such as finely dispersed silicon dioxide (Aerosil® from Evonik Industries). The dried microcapsule dispersion (powder mixture) containing the emulsifier can then be incorporated into products, intermediates, or formulations, for example, by redispersing in a liquid medium, preferably in an aqueous phase. In this way, the dry content of the formulation can be directly adjusted by the mixing ratio of the powder mixture and the liquid medium.

### Use of the Microcapsule Dispersion and Product

According to a third aspect, the invention relates to a product containing a microcapsule dispersion according to the first aspect.

The phase-stabilizing effect of the emulsifier on the microcapsules is particularly significant when the microcapsule dispersion is brought into contact with or mixed with another solution or dispersion during the manufacture of a product, forming an intermediate product or the final product. The product can be solid or liquid. According to one embodiment, the product is liquid.

The phase-stabilizing effect occurs over a wide pH range. In particular, the phase-stabilizing effect of the emulsifier is effective at a non-strongly basic pH value. According to one embodiment, the pH of the product is less than 10. For example, the pH value of the product can be 9.5, 9.0, 8.5, 8.0, 7.5, 7.0, 6.5, 6.0, 5.5, 5.0, 4.5, 4.0, 3.5, 3.0, 2.5, 2.0, or 1.5. The pH value can be less than 9, less than 8, or less than 7. The phase-stabilizing effect of the emulsifier is particularly significant in an acidic environment. Therefore, in one embodiment, the pH value of the product is less than 6, preferably less than 5, more preferably less than 4, and particularly preferably less than 3.

The phase-stabilizing effect occurs over a wide conductivity range. According to one embodiment, the conductivity of the product is up to 100 mS/cm, preferably up to 60 mS/cm, up to 50 mS/cm, or up to 40 mS/cm, particularly preferably 34 mS/cm, with typical conductivities ranging from at least 0.1 mS/cm, at least 0.2 mS/cm, at least 0.3 mS/cm, at least 2.0 mS/cm, or at least 8.0 mS/cm. For example, the conductivity can be 0.4 mS/cm, 0.5 mS/cm, 0.6 mS/cm, 0.7 mS/cm, 0.8 mS/cm, 0.9 mS/cm, 1.0 mS/cm, 1.5 mS/cm, 2.0 mS/cm, 3.0 mS/cm, 4.0 mS/cm, 5.0 mS/cm, 6.0 mS/cm, 7.0 mS/cm, 8.0 mS/cm, 9.0 mS/cm, 10.0 mS/cm, 12.0 mS/cm, 14.0 mS/cm, 16.0 mS/cm, 18.0 mS/cm, 20.0 mS/cm, 22.0 mS/cm, 24.0 mS/cm, 26.0 mS/cm, 28.0 mS/cm, 30.0 mS/cm, 32.0 mS/cm, or 34.0 mS/cm. According to one embodiment of the product, the conductivity is in the range of 0.2 mS/cm to 6.0 mS/cm, preferably in the range of 0.3 mS/cm to 5.0 mS/cm, more preferably in the range of 0.4 mS/cm to 4.0 mS/cm. In this case, the pH is preferably in the acidic range, particularly below pH 4.0. According to another embodiment, the conductivity is in the range of 7.0 mS/cm to 40.0 mS/cm, preferably in the range of 8.0 mS/cm to 34.0 mS/cm. In this case, the pH is preferably in the neutral or basic range, particularly in the range of 7.5 to 9.0.

Due to the robustness and impermeability of these biodegradable capsules, the microcapsule dispersions can be used in a variety of products.

The product can be an adhesive system; a pharmaceutical product; a coating material, particularly a coated paper; a heat-storage coating, a self-healing coating, or a corrosion protection coating; or a microcapsule-containing coating for functional packaging materials.

Regardless of the type of products, these comprise the microcapsules described herein as well as the emulsifier described herein, with these two components being pre-formulated as microcapsule dispersions, i.e., brought into contact before being added to the product according to the invention, typically by pre-formulating the capsules in a slurry containing the emulsifier. Depending on the amount of microcapsules used in the final product, the amount of the co-formulated emulsifier in the microcapsule slurry also varies. Typical amounts range from 0.001 wt. % to 0.25 wt. % based on the total weight of the product. Increasingly preferred ranges are up to 0.20 wt. %, up to 0.15 wt. %, up to 0.12 wt. %, up to 0.10 wt. %, or up to 0.08 wt. %. The lower limit is typically 0.001 wt. %, 0.005 wt. %, or 0.01 wt. %. It has been found that the stabilizing effect of the emulsifiers used extends not only to the pre-formulated slurry but also to the (liquid) final product, so that the phase-stabilizing effect on the microcapsules can also be observed in the final product. However, this effect depends on the pre-formulation of the microcapsules with the emulsifier and does not occur when microcapsules and emulsifier are formulated separately into the product.

Furthermore, according to a fourth aspect, the invention relates to the use of microcapsules according to the first aspect for the production of a product. In other words, the microcapsules can be used in the production of a product according to the third aspect. The microcapsule dispersion can be used to form the product or its intermediate product. According to one embodiment, the final product or the intermediate product formed by the addition of the microcapsule dispersion has a pH value and/or conductivity as defined for the product according to the third aspect.

According to one embodiment, the final product or the intermediate product formed by the addition of the microcapsule dispersion has a pH value of less than 10, preferably less than 9, more preferably less than 5, and particularly preferably less than 4 and/or a conductivity of more than 0.3 mS/cm, preferably more than 1.0 mS/cm, more preferably more than 2.5 mS/cm, and particularly preferably more than 5.0 mS/cm.

According to one embodiment of the use according to the fourth aspect, the product is selected from the group consisting of an adhesive system; a pharmaceutical product; a coating material, particularly a coated paper; a heat storage coating, a self-healing coating, or a corrosion protection coating; and such microcapsule-containing coatings for functional packaging materials.

### Manufacturing Process

Methods for producing core/shell microcapsules are known to the skilled person. Typically, an oil-based core material that is not or only slightly water-soluble is emulsified or dispersed in an aqueous phase containing the wall-forming agents. Depending on the viscosity of the liquid core materials, various devices, ranging from simple stirrers to high-performance dispersers, are used to distribute the core material into fine oil droplets. The wall-forming agents precipitate from the continuous water phase onto the surface of the oil droplets and can then be cross-linked.

This mechanism is used in the in-situ polymerization of amino and phenoplast microcapsules and in the coacervation of water-soluble hydrocolloids.

In contrast, oil-soluble acrylate monomers are used for wall formation in radical polymerization. Furthermore, methods are used in which water-soluble and oil-soluble starting materials are brought to react at the phase boundary of the emulsion droplets to form the solid shell.

Examples are the reaction of isocyanates and amines or alcohols to form polyurea or polyurethane walls (interfacial polymerization), as well as the hydrolysis of silicate precursors with subsequent condensation to form an inorganic capsule wall (sol-gel process).

In suitable methods for producing microcapsules comprising a fragrance as the core material and a shell consisting of three layers, the barrier layer serving as a diffusion barrier is used as a template. Very small amounts of the aforementioned wall-forming agents are required to build this barrier layer. Preferably, after droplet formation at high stirring speeds, the sensitive templates are equipped with an electrically negative charge by suitable protective colloids (e.g., Poly-AMPS) so that neither Ostwald ripening nor coalescence can occur. After producing this stable emulsion, the wall-forming agent, for example, a suitable precondensate based on amino resin, can form a very thin shell (layer) at a significantly reduced stirring speed. The thickness of the shell can be further reduced, particularly by the addition of an aromatic alcohol, such as m-aminophenol. This is followed by the formation of a production-ready shell structure, which unexpectedly shows a good affinity for biopolymers such as gelatin or alginate upon addition and deposits on the templates without the expected problems such as gelation of the mixture, agglomeration formation, and incompatibility of the structure-forming agent.

The use of the emulsion stabilizer can further increase the deposition of the biopolymers.

According to a second aspect, the invention relates to a method for producing the microcapsule dispersion according to the invention. The method comprises at least the following steps:
a) preparing an oil-in-water emulsion by emulsifying a core material in an aqueous phase in the presence of the wall-forming component(s) of the inner barrier layer with the addition of protective colloids;
b) depositing and curing the wall-forming component(s) of the barrier layer, wherein the wall-forming component(s) of the barrier layer preferably are an aldehydic component, an amine component, and an aromatic alcohol, particularly preferably formaldehyde, melamine, and resorcinol;
c) optionally adding an emulsion stabilizer, where the emulsion stabilizer is as defined herein:
d) adding the wall-forming component(s) of the stability layer, followed by deposition and curing, where the wall-forming component(s) of the stability layer are at least one biopolymer, preferably a protein and/or a polysaccharide, particularly preferably gelatin and alginate, as well as a curing agent, preferably glutaraldehyde or glyoxal; and
e) optionally adding the wall-forming component(s) of the outer, third shell layer, followed by deposition and curing, wherein the wall-forming component(s) of the outer, third shell layer are preferably an amine component, particularly melamine.

The addition of the emulsion stabilizer is preferably done slowly over at least two minutes. According to one embodiment, the microcapsule dispersion is stirred. For stirring, a paddle stirrer can be used, for example. The stirring speed is preferably in the range of 150 to 250 rpm. Above 250 rpm, there is a risk of air being introduced into the microcapsule dispersion. Below 150 rpm, the mixing might not be sufficient.

The temperature is preferably in the range of 15° C. to 35° C. The temperature can be 15° C., 18° C., 20° C., 23° C., 25° C., 28° C., 30° C., 33° C., or 35° C. Particularly preferred is a temperature of 25° C. After addition, the microcapsule dispersion is stirred until a homogeneous mixture is formed. In one embodiment, the microcapsule dispersion is stirred for at least 5 minutes after the addition. In a preferred embodiment, the microcapsule dispersion is stirred for at least 10 minutes after the addition.

Alternatively, steps a) and b) can be performed as follows:
a) preparing an oil-in-water emulsion by emulsifying a core material in an aqueous phase in the presence of the wall-forming component(s) of the inner barrier layer, optionally with the addition of protective colloids;
b) depositing and curing the wall-forming component(s) of the inner barrier layer, wherein the wall-forming component(s) of the inner barrier layer particularly are an aldehydic component, an amine component, and an aromatic alcohol.

This method can be conducted either sequentially or as a so-called one-pot process. In the sequential process, only steps a) and b) are carried out in a first procedure until microcapsules with only the inner barrier layer as the shell (intermediate microcapsules) are obtained. Subsequently, a portion or the entire amount of these intermediate microcapsules is transferred to another reactor. The further reaction steps are then carried out in this reactor. In the one-pot process, all process steps are performed in a single batch reactor. Conducting the process without changing reactors is particularly time-saving.

The entire system should be tuned to the one-pot process for this purpose. The correct choice of solid content, appropriate temperature control, coordinated addition of formulation components, and sequential addition of wall-formers can be achieved in this way.

In one embodiment of the method, the method comprises the preparation of an aqueous phase by dissolving a protective colloid, particularly a polymer based on acrylamidosulfonate and a methylated pre-polymer in water. The pre-polymer is preferably produced by reacting an aldehyde with either melamine or urea. Optionally, methanol can be used in this method.

Furthermore, the method can include mixing the aqueous phase by stirring and setting an initial temperature, with the initial temperature in the range of 30° C. to 40° C. Subsequently, an aromatic alcohol, particularly phloroglucinol, resorcinol, or aminophenol, can be added to and dissolved in the aqueous phase.

Alternatively, the method can include preparing an oil phase by mixing a fragrance composition or a phase change material (PCM) with aromatic alcohols, particularly phloroglucinol, resorcinol, or aminophenol. Alternatively, reactive monomers or diisocyanate derivatives can also be incorporated into the fragrance composition. Subsequently, the first temperature can be set.

Another step can be the preparation of a two-phase mixture by adding the oil phase to the aqueous phase and then increasing the stirring speed.

Subsequently, emulsification can be initiated by adding formic acid. Regular determination of the particle size is advisable. Once the desired particle size is reached, the two-phase mixture can be further stirred while setting a second temperature for curing the capsule walls. The second temperature can be in the range of 55° C. to 65° C.

Subsequently, a melamine dispersion can be added to the microcapsule dispersion, and a third temperature can be set, preferably in the range of 75° C. to 85° C.

Another suitable step is the addition of an aqueous urea solution to the microcapsule dispersion. Subsequently, the emulsion stabilizer is added to the microcapsule dispersion before it is added to a solution of gelatin and alginate to form the stability layer.

In this case, the microcapsule dispersion would then be cooled to 45° C. to 55° C., and the pH of the microcapsule dispersion would be adjusted to a value in the range of 3.5 to 4.1, particularly 3.7.

The microcapsule dispersion can then be cooled to a fourth temperature in the range of 20° C. to 30° C. It can then be further cooled to a fifth temperature, which is in the range of 4° C. to 17° C., particularly 8° C.

Subsequently, the pH of the microcapsule dispersion would be adjusted to a value in the range of 4.3 to 5.1, and glutaraldehyde or glyoxal would be added. The reaction conditions, particularly temperature and pH, can be chosen differently depending on the cross-linker. The appropriate conditions can be determined by the skilled person, for example, based on the reactivity of the cross-linker. The amount of glutaraldehyde or glyoxal added influences the cross-linking density of the first layer (stability layer) and thus, for example, the impermeability and biodegradability of the microcapsule shell. Accordingly, the skilled person can vary the amount specifically to adjust the property profile of the microcapsule. To create the additional third layer, a melamine suspension consisting of melamine, formic acid, and water can be prepared. This is followed by the addition of the melamine suspension to the microcapsule dispersion. Finally, the pH of the microcapsule dispersion would be adjusted to a value in the range of 9 to 12, particularly 10 to 11.

Furthermore, the microcapsule dispersion can be heated to a temperature in the range of 20° C. to 80° C. for curing in step e). This temperature can have an influence on the color stability of the microcapsules. The temperature can be 20° C., 25° C., 30° C., 35° C., 40° C., 45° C., 50° C., 55° C., 60° C., 65° C., 70° C., 75° C., or 80° C. Below a temperature of 20° C., no influence on the color stability is expected. A temperature above 80° C. could negatively affect the properties of the microcapsules. According to one embodiment, the temperature is in the range of 30° C. to 60° C. According to a preferred embodiment, the temperature is in the range of 35° C. to 50° C.

According to one embodiment, the microcapsule dispersion is maintained at the heating temperature for a period of at least 5 minutes. The period can be, for example, 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 100 minutes, 110 minutes, or 120 minutes. According to one embodiment, the microcapsule dispersion is maintained at the heating temperature for a period of at least 30 minutes. According to one embodiment, the microcapsule dispersion is maintained at the heating temperature for a period of at least 60 minutes.

In one process step of producing the microcapsule dispersion, the emulsifier selected from the group of ethoxylated, hydrogenated castor oils is added. Preferably, the emulsifier, such as Eumulgin® HRE 40, is added after the curing step e). In this process, the microcapsules are typically dispersed into an aqueous continuous phase that already contains the emulsifier used according to the invention, thereby creating the slurries according to the invention. The amounts/concentrations of emulsifier, microcapsules, and water used are as defined above.

As an alternative to using the emulsifier, i.e., the hydrogenated, ethoxylated castor oil, an improvement in the phase stability of dispersions of the microcapsules according to the invention in a fabric softener base can also be achieved by pre-dilution of the microcapsule dispersion.

Accordingly, the invention further relates to the dilution of a microcapsule dispersion containing biodegradable microcapsules comprising a core material and a shell, wherein the shell consists of at least one barrier layer and one stability layer, wherein the barrier layer surrounds the core material, wherein the stability layer comprises at least one biopolymer and is arranged on the outer surface of the barrier layer, and optionally an emulsion stabilizer is arranged at the transition from the barrier layer to the stability layer; in an aqueous solution with a minimum ratio of the aqueous solution to the microcapsule dispersion of 1:99.

The microcapsule dispersion can be diluted with an aqueous solution at a ratio of aqueous solution to microcapsule dispersion of 1:19, 1:15, 1:13, 1:11, 1:9, 1:7, 1:5, 1:4, 1:3, 1:2, 1:1, 2:1, 3:1, 4:1, 5:1, 7:1, 9:1, 11:1, 13:1, 15:1, or 19:1. According to one embodiment, the microcapsule dispersion is diluted to a ratio of aqueous solution to microcapsule dispersion in the range of 1:15 to 9:1. According to one embodiment, the microcapsule dispersion is diluted to a ratio of aqueous solution to microcapsule dispersion in the range of 1:9 to 5:1. According to one embodiment, the microcapsule dispersion is diluted to a ratio of aqueous solution to microcapsule dispersion in the range of 1:3 to 2:1.

Furthermore, according to an alternative form of the second aspect, the invention relates to a method for producing a microcapsule dispersion, comprising the steps:
a) preparing an oil-in-water emulsion by emulsifying a core material in an aqueous phase in the presence of the wall-forming component(s) of the inner barrier layer with the addition of protective colloids;
b) depositing and curing the wall-forming component(s) of the barrier layer, wherein the wall-forming component(s) of the barrier layer preferably are an aldehyde component, an amine component, and an aromatic alcohol, more preferably formaldehyde, melamine, and resorcinol;
c) optionally adding an emulsion stabilizer, wherein the emulsion stabilizer is as defined herein;
d) adding the wall-forming component(s) of the stability layer, followed by deposition and curing, wherein the wall-forming component(s) of the stability layer are at least one biopolymer, preferably a protein and/or a polysaccharide, more preferably gelatin and alginate, and a curing agent, preferably glutaraldehyde or glyoxal; and
e) optionally adding the wall-forming component(s) of the outer, third shell layer, followed by deposition and curing, wherein the wall-forming component(s) of the outer, third shell layer preferably are an amine component, particularly melamine.
f) diluting the microcapsule dispersion to a microcapsule concentration in the range of 0.1 wt. % to 50 wt. % in an aqueous solution.

According to one embodiment, the aqueous solution consists of water. The temperature of the aqueous solution ranges from 1° C. to 100° C. The temperature when introduced into the product can be, for example, 1° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C. 95° C., or 100° C. According to one embodiment, the temperature of the aqueous solution ranges from 15° C. to 90° C. According to one embodiment, the temperature of the aqueous solution ranges from 30° C. to 80° C. According to one embodiment, the temperature of the aqueous solution ranges from 40° C. to 80° C.

According to one embodiment, the temperature of the aqueous solution ranges from 50° C. to 70° C. According to one embodiment, the temperature of the aqueous solution is about 60° C.

Preferably, the microcapsule dispersion is diluted shortly after production, i.e., after the completion of step d) or e). The time between step d) or e) and dilution f) is at most six months. The time between step d) or e) and dilution f) can be, for example, 5 min, 30 min, 1 h, 2 h, 4 h, 6 h, 8 h, 10 h, 12 h, 18 h, 24 h, 2 days, 4 days, 7 days, 2 weeks, 3 weeks, 4 weeks, 2 months, 3 months, 4 months, 5 months, or 6 months. According to one embodiment, the time between step d) or e) and dilution f) is at most 1 week.

According to one embodiment, the time between step d) or e) and dilution f) is at most 2 days.

The diluted microcapsule concentration can be 0.1 wt. %, 0.2 wt. %, 0.5 wt. %, 0.8 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 25 wt. %, 30 wt. %, 35 wt. %, 40 wt. %, 45 wt. % or 50 wt. %.

According to one embodiment, the microcapsule concentration ranges from 5 wt. % to 30 wt. %. According to one embodiment, the microcapsule concentration ranges from 8 wt. % to 20 wt. %. According to one embodiment, the microcapsule concentration ranges from 10 wt. % to 15 wt. %.

Although the dilution of the microcapsule dispersion before addition to the fabric softener base (pre-dilution) does not quite achieve the phase stability obtained with the hydrogenated, ethoxylated castor oil, the phase stability is still significantly better than with dosing without pretreatment.

Preferably, the diluted microcapsule dispersion is added to the product for which it is intended while still warm. Consequently, the invention also relates to a method for producing a product comprising the steps for producing the microcapsule dispersion and the additional step g) introducing the microcapsule dispersion into the product.

Methods for mixing microcapsule dispersions into final products are known to the skilled person. Preferably, the microcapsule dispersion is introduced into the product shortly after dilution. The time between dilution and introduction into the product is at most six months. The time between dilution and introduction into the product can be, for example, 5 min, 30 min, 1 h, 2 h, 4 h, 6 h, 8 h, 10 h, 12 h, 18 h, 24 h, 36 h, 2 days, 4 days, 7 days, 2 weeks, 3 weeks, 4 weeks, 6 weeks, 2 months, 3 months, 4 months, 5 months, or 6 months. According to one embodiment, the time between dilution and introduction into the product is at most one week. According to one embodiment, the time between dilution and introduction into the product is at most two days.

Preferably, the microcapsule dispersion is introduced into the product while still warm. The temperature when introducing into the product is at least 20° C. The temperature when introducing into the product can be, for example, 20° C., 25° C., 30° C., 35° C., 40° C., 45° C., 50° C., 55° C., 60° C., 65° C., 70° C., 75° C., 80° C., 85° C., 90° C., 95° C., or 100° C. According to one embodiment, the temperature when introducing into the product ranges from 20° C. to 90° C. According to one embodiment, the temperature of the aqueous solution ranges from 25° C. to 70° C. According to one embodiment, the temperature of the aqueous solution ranges from 30° C. to 60° C. According to one embodiment, the temperature of the aqueous solution ranges from 40° C. to 50° C. According to one embodiment, the temperature of the aqueous solution is about 45° C.

It is also possible, after pre-dilution, to add the emulsifier according to the invention (hydrogenated, ethoxylated castor oil) to the microcapsule dispersion before it is then added to the final product, for example, a fabric softener. This can further improve phase stability. According to one embodiment, the emulsifier is added to the pre-diluted microcapsule dispersion in a concentration ranging from 0.1 wt. % to 50 wt. %.

Accordingly, the invention further relates to a microcapsule dispersion containing:
biodegradable microcapsules comprising a core material and a shell, wherein the shell consists of at least one barrier layer and a stability layer, wherein the barrier layer surrounds the core material, wherein the stability layer comprises at least one biopolymer and is arranged on the outer surface of the barrier layer, and wherein optionally an emulsion stabilizer is arranged at the transition from the barrier layer to the stability layer;
produced by a method according to the second aspect, wherein, after step d) or e), the microcapsule dispersion is diluted in an aqueous solution to a microcapsule concentration ranging from 0.1 wt. % to 50 wt. %

According to one embodiment of the microcapsule dispersion, no emulsifier is added in the production process of the microcapsule dispersion. According to one embodiment of the microcapsule dispersion, the emulsifier according to the invention is added in the production process of the microcapsule dispersion after dilution f).

### Microcapsule Dispersion and Coloration

Despite the use of aromatic alcohol in the barrier layer of the microcapsule shell, the microcapsule dispersions according to the invention show only slight coloring.

To qualify the discoloration, the color coordinate in the L*a*b* color space was determined for the microcapsules according to the invention. The L*a*b* color model is standardized in EN ISO 11664-4 “Colorimetry—Part 4: CIE 1976 L*a*b* Colour space”. The L*a*b* color space (also known as CIELAB, CIEL*a*b*, Lab colors) describes all perceptible colors. It uses a three-dimensional color space, where the brightness value L* is perpendicular to the color plane (a*,b*). The a-coordinate indicates the color type and color intensity between green and red, and the b-coordinate indicates the color type and color intensity between blue and yellow. The larger the positive a- and b-values and the smaller the negative a- and b-values, the more intense the color tone. If a=0 and b=0, it is an achromatic color tone on the brightness axis. The properties of the L*a*b* color model include device independence and perception relevance, meaning colors are defined regardless of their method of production or reproduction technique, as they are perceived by a standard observer under standard lighting conditions.

The microcapsule dispersions according to the invention in the L*a*b* color space have a color coordinate with an L* value of at least 50. The L* value can be, for example, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80. According to a preferred embodiment, the microcapsule dispersions according to the invention in the L*a*b* color space have a color coordinate with an L* value of at least 50. Particularly preferred is a color coordinate of at least 60.

Furthermore, the microcapsule dispersions produced by the manufacturing method according to the invention are particularly color-stable. The color coordinate of the microcapsule dispersion in the L*a*b* color space after storage has an L* value of at least 50. The L* value after storage can be, for example, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80.

According to a preferred embodiment, the microcapsule dispersions according to the invention have a color coordinate in the L*a*b* color space with an L* value of at least 60 after storage. Particularly preferred is a color coordinate of at least 65.

According to one embodiment, the storage time is at least four weeks, preferably at least six weeks, and particularly at least eight weeks.

### EXAMPLES

The microcapsule dispersions according to the invention in the following examples all contain 5 wt. % emulsifier from the group of ethoxylated, hydrogenated castor oils with an EQ value of 40 (slurry=suspension), particularly Eumulgin® HRE 40. The emulsifier was added to the microcapsule dispersions after prior melting particularly directly before dosing into the fabric softener base.

### Example 1—Production of Reference Microcapsules with Melamine-Formaldehyde Recipe

### 1.1 Materials

The materials used for the production of the reference microcapsules—melamine-formaldehyde—are shown in Table 1.

TABLE 1
List of substances used for the production of MK2

| | | Concentration/ | MK2 |
|---|---|---|---|
| Trade Name | Substance Name | wt. % | Weight/g |
| — | Deionized water | 100 | 187.5 |
| Dimension ™ | 1,3,5-Triazine- | 67 | 42.5* |
| SD, Solenis | 2,4,6-triamine, | | |
| | polymer with | | |
| | formaldehyde, | | |
| | methylated | | |
| | (content (w/w): | | |
| | >=60% − <=80%), | | |
| | in water | | |
| Dimension ™ | Polymer based on | 20 | 35.0* |
| SD, Solenis | acrylamidosulfonate | | |
| Weiroclean, | Core material | 100 | 192.5* |
| Kurt Kitzing | (e.g., fragrance | | |
| GmbH | oil, PCM, etc.) | | |
| — | Formic acid | 10 | 8.8 |
| Melafine ®, | Melamine | | 48.8 |
| OCI Nitrogen | suspension ¹⁾ | 27 | |
| B.V. | | | |
| — | Urea solution | 28.6 | 70.0 |

¹⁾ Concentration refers to the acidified suspension..
*Quantities of the components refer to the commercial product and are used as supplied.

### 1.2 Manufacturing Process (Based on BASF Patent EP 1 246693 B1)

Dimension SD was stirred into deionized water, after which Dimension PA140 was added and stirred until a clear solution was formed. The solution was heated to 30-35° C. in a water bath. While stirring with a dissolver disc, the perfume oil was added at 1100 rpm. The pH value of the oil-in-water emulsion was adjusted to 3.3-3.8 with a 10% formic acid solution. Then, the emulsion was stirred for another 30 minutes at 1100 rpm until a droplet size of 20-30 μm was reached, or the stirring was extended until the desired particle size of 20-30 μm (Peak-Max) was achieved. The particle size was determined using a Beckmann-Coulter device (laser diffraction, Fraunhofer method). The rotation speed was reduced depending on the viscosity to ensure good mixing. With this rotation speed, the mixture was stirred for another 30 minutes at 30 to 40° C. Subsequently, the emulsion was heated to 60° C. and stirred further. The melamine suspension was adjusted to a pH value of 4.5 with formic acid (10%) and added to the reaction mixture. The mixture was maintained at 60° C. for 60 minutes and then heated to 80° C. After stirring for 60 minutes at 80° C., the urea solution was added.

After cooling to room temperature, the microcapsule dispersion was filtered through a 200-μm filter sieve.

### Example 2—Production of Microcapsule Dispersions According the Invention Slurry 2 and Slurry 5 and Reference Microcapsule not According to the Invention MK1

### 2.1 Materials

The materials used for the production of the microcapsules according to the invention—Slurry 2 and Slurry 5 are shown in Table 2.

TABLE 2
List of substances used for the production of Slurry 2 and 5

| | | | Slurry | Slurry |
|---|---|---|---|---|
| | | Concen- | 2 | 5 |
| | | tration/ | Weight/ | Weight/ |
| Trade Name | Substance Name | wt. % | g | g |
| | Deionized water | 100 | 35.2 | 39.6 |
| | addition 1 | | | |
| Dimension ™ | 1,3,5-Triazine- | 67 | 2.1* | 1.7* |
| SD, Solenis | 2,4,6-triamine, | | | |
| | polymer with | | | |
| | formaldehyde, | | | |
| | methylated | | | |
| | (content (w/w): | | | |
| | >=60% − <=80%), | | | |
| | in water | | | |
| Dimension ™ | Polymer based on | 20 | 4.6* | 3.8* |
| PA 140, Solenis | acrylamidosulfonate | | | |
| | addition 1 | | | |
| Weiroclean, Kurt | Core material | 100 | 52.8* | 52.6* |
| Kitzing GmbH | (e.g., fragrance oil, | | | |
| | PCM, etc.) | | | |
| — | Resorcinol solution | 3.2 | 12.7 | 11.9 |
| — | Fomic acid addition | 10 | 0.7 | 0.4 |
| | 1 | | | |
| Melafine ®, OCI | Melamine | 27 | 2.6 | 2.0 |
| Nitrogen B.V. | suspension | | | |
| | addition 1 ¹⁾ | | | |
| — | Urea solution | 41.9 | 2.6 | 2.2 |
| Dimension ™ | Polymer based on | 20 | 9.5* | 9.6* |
| PA 140, Solenis | acrylamidosulfonate | | | |
| | addition 2 | | | |
| — | Tap water | 100 | 136.2 | 135.6 |
| — | Sodium sulfate | 100 | 0.6* | 0.6* |
| Scogin ® MV, | Sodium alginate | 100 | 2.0* | 2.0* |
| DuPont Nutrition | | | | |
| Ireland | | | | |
| Speisegelatinepulver, | Pork skin gelatin | 100 | 8.4* | 8.4* |
| Ewald-Gelatine | | | | |
| GmbH | | | | |
| — | Formic acid | 20 | 2.5 | 2.5 |
| | addition 2 | | | |
| — | Sodium hydroxide | 20 | 2.4 | 2.3 |
| | Addition 1 ²⁾ | | | |
| Glutaraldehyde, | Glutaraldehyde | 50 | 2.6* | 2.6* |
| 50% aq. soln., | solution | | | |
| Alfa Aesar | | | | |
| Melafine ®, OCI | Melamine | 27 | 27.5 | 27.4 |
| Nitrogen B.V. | suspension | | | |
| | addition 2 ¹⁾ | | | |
| — | Sodium hydroxide | 20 | 4.7 | 4.7 |
| | addition 2 | | | |

¹⁾ Concentration refers to the acidified suspension..
²⁾ The quantities given for acids/bases are guide values. It is adjusted to the pH range specified in the experimental procedure.
*Quantities of the components refer to the commercial product and are used as supplied.

The materials used for the production of the reference microcapsules MK1 are shown in Table 3.

TABLE 3
List of substances used for the production of MK1

| | | MK1 | |
|---|---|---|---|
| | | Concen- | |
| | | tration/ | Weight/ |
| Trade Name | Substance Name | wt. % | g |
| | Deionized water addition 1 | 100 | 34.9 |
| Dimension ™ SD, | 1,3,5-Triazine-2,4,6- | 67 | 1.6* |
| Solenis | triamine, polymer with | | |
| | formaldehyde, methylated | | |
| | (Content (W/W): | | |
| | >=60% − <=80%), | | |
| | in water | | |
| Dimension ™ PA | Polymer based on | 20 | 3.4* |
| 140, Fa. Solenis | acrylamidosulfonate | | |
| Weiroclean, Kurt | Core material (e.g., | 100 | 38.8* |
| Kitzing GmbH | fragrance oil, PCM, etc.) | | |
| — | Resorcinol solution | 12.2 | 2.5 |
| — | Formic acid addition 1 | 20 | 0.5 |
| Melafine ®, OCI | Melamine suspension | 27 | 1.9 |
| Nitrogen B.V. | addition 1 ¹⁾ | | |
| — | Urea solution | 16.6 | 4.7 |
| — | Tap water | 100 | 100.19 |
| — | Sodium sulfate | 100 | 0.5* |
| Speisegelatinepulver, | Pork skin gelatin | 100 | 6.2* |
| Ewald-Gelatine GmbH | | | |
| Scogin ® MV, DuPont | Sodium alginate | 100 | 1.4* |
| Nutrition Ireland | | | |
| — | Formic acid addition 2 | 20 | 1.4 |
| — | Sodium hydroxide | 20 | 0.8 |
| | addition 1 ²⁾ | | |
| Glutaraldehyde, 50% | Glutaraldehyde solution | 50 | 1.9* |
| aq. soln., Alfa Aesar | | | |
| Melafine ®, OCI | Melamine suspension | 27 | 6.7 |
| Nitrogen B.V. | addition 2 ¹⁾ | | |
| | Sodium hydroxide | 20 | 2.2 |
| | addition 2 | | |

¹⁾ Concentration refers to the acidified suspension..
²⁾ The quantities given for acids/bases are guide values. It is adjusted to the pH range specified in the experimental procedure.
*Quantities of the components refer to the commercial product and are used as supplied.

### 2.2 Manufacturing Process for the Microcapsule not According to the Invention MK1

To prepare reaction mixture 1, Dimension PA140 and Dimension SD were weighed with deionized water (addition 1) in a beaker and pre-mixed using a 4 cm dissolver disc. The beaker was fixed in a water bath and stirred at 500 RPM at 30° C. with the dissolver disc until a clear solution was formed.

Once the Dimension SD/Dimension PA140 solution became clear and reached 30-40° C., the perfume oil was slowly added while adjusting the stirring speed (1100 RPM) to achieve the desired particle size. The pH of this mixture was then acidified by adding formic acid (addition 1). The mixture was emulsified for 20-30 minutes, or extended as needed until the desired particle size of 20-30 μm (Peak-Max) was reached. The particle size was determined using a Beckman-Coulter device (laser diffraction, Fraunhofer method). After reaching the desired particle size, the stirring speed was reduced to ensure gentle mixing.

The resorcin solution was then stirred in and pre-formed gently for 30-40 minutes. After the pre-formation time, the emulsion temperature was raised to 50° C. within 15 minutes. Upon reaching this temperature, the mixture was heated to 60° C. over 15 minutes and maintained at this temperature for another 30 minutes. Then, using 20% formic acid, the pH of melamine suspension addition 1 was adjusted to 4.5 and dosed into the reaction mixture over 90 minutes. The temperature was maintained for 30 minutes. After this period, the temperature was first raised to 70° C. within 15 minutes, then to 80° C. within another 15 minutes, and held for 120 minutes. Afterward, the aqueous urea solution was added, the heat source turned off, and Reaction Mixture 1 cooled to room temperature. In a separate beaker, sodium sulfate was dissolved in tap water while stirring with a wing stirrer at 40-50° C. Sodium alginate and pork skin gelatin were slowly sprinkled into the heated water. After all solids dissolved, reaction mixture 1 was stirred into the prepared gelatin/sodium alginate solution. Once a homogeneous mixture was reached, the pH was adjusted to 3.9 with formic acid addition 2, and then the heat source was removed. The mixture was then cooled to room temperature. After reaching room temperature, the reaction mixture was cooled with ice. Upon reaching 8° C., the ice bath was removed, and the pH was raised to 4.7 with Sodium hydroxide addition 1. Glutaraldehyde was then added, ensuring that the temperature did not exceed 16-20° C. until its addition.

Then, the melamine suspension addition 2, acidified to a pH of 4.5 with 20% formic acid, was dosed slowly. The reaction mixture was then heated to 60° C. and maintained at this temperature for 60 minutes. After this holding time, the heat source was removed, and the microcapsule suspension was gently stirred for 14 hours. After 14 hours, the microcapsule suspension was adjusted to a pH of 10.5 with sodium hydroxide addition 2.

### 2.3 Manufacturing Process for the Microcapsule Dispersions According to the Invention Slurry 2 Und Slurry 5

To prepare reaction mixture 1, Dimension PA140 addition 1 and Dimension SD were weighed with deionized water addition 1 in a beaker and pre-mixed with a 4 cm dissolver disc. The beaker was fixed in a water bath, and stirred with the dissolver disc at 500 rpm at 30° C. until a clear solution was formed.

As soon as the Dimension SD/Dimension PA140 solution was clear and reached 30-40° C., the core material was slowly added while adjusting the speed (e.g., 1100 rpm) to achieve the desired particle size. Then, the pH value of this mixture was acidified to by adding formic acid addition 1 (pH=3.3-3.5).

It was emulsified for 20-30 min or extended until the desired particle size of 20-30 μm (Peak-Max) was reached. The particle size was determined using a Beckmann-Coulter device (laser diffraction, Fraunhofer method). After reaching the particle size, the speed was reduced to ensure gentle mixing and the resorcinol solution was added.

Under gentle stirring, it was preformed for 30-40 min. After the preforming time elapsed, the emulsion temperature was raised to 50° C. within 15 min. Upon reaching this temperature, the mixture was heated to 60° C. over a period of 15 min and this temperature was maintained for another 30 min. Then, the pH value of the melanine suspension addition 1 was adjusted to 4.5 with 20% formic acid and added to the reaction mixture over a period of 90 min.

After that, the temperature was maintained for 30 min. After the 30 min, the temperature was first raised to 70° C. within 15 min. Then, the temperature was raised to 80° C. within another 15 min and maintained for 90 min.

Then, the aqueous urea solution was added, the heat source was turned off, and reaction mixture 1 was cooled to room temperature. After reaction mixture 1 reached room temperature, Dimension PA140 addition 2 was added.

In a separate beaker, sodium sulfate was dissolved in tap water under stirring with a paddle stirrer at 40-50° C. Sodium alginate and pork skin gelatin were slowly sprinkled into the heated tap water. After all solids were dissolved, reaction mixture 1 was stirred into the prepared gelatin/sodium alginate solution. When a homogeneous mixture was achieved, the pH value was adjusted to 3.7 by slowly adding formic acid addition 2, then the heat source was removed, and the mixture was naturally cooled to room temperature.

After reaching room temperature, the reaction mixture was cooled with ice. Upon reaching a temperature of 8° C., the ice bath was removed, and the pH value was raised to 4.7 with sodium hydroxide addition 1. Then, glutaraldehyde 50% was added, ensuring that the temperature did not exceed 16-20° C. until the addition of glutaraldehyde 50%.

Subsequently, the melamine suspension addition 2, acidified to a pH of 4.5 with 20% formic acid, was added over a period of about 2 min. The microcapsule suspension was then gently stirred at room temperature for 14 hours. After the 14 hours, the microcapsule suspension was adjusted to a pH of 10.5 with sodium hydroxide addition 2 over a period of about 15 min.

### Example 3—Stability

The stability of the capsules described herein when used in capsule slurries and commercially available fabric softener formulations was investigated. As a comparison, commercially available melamine-formaldehyde capsules (ME capsules) and the capsules according to PCT/EP2020/085804, which do not have an emulsion stabilizer between the inner and outer shell, were used.

To evaluate the phase stability of the microcapsule slurry and the microcapsules in a final product(fabric softener formulation), corresponding formulations with the addition of the different perfume microcapsules (0.3 wt. % of a capsule slurry with equal amounts of capsules) were formulated and stored for 4 weeks at room temperature (20-25° C.). The stability was evaluated on the following scale: 1=no phase separation, 2=slight phase separation, 3=medium phase separation, 4=strong phase separation and 5=very strong phase separation. The results are shown in Table 4 below.

TABLE 4
Phase stability of the capsules after 4 weeks of storage at
room temperature

| | |
|---|---|
| | Phase stability |
| | after 4 weeks |
| | at room |
| Product | temperature |

Fabric softener (0.3 wt. % capsule slurry each)

| | |
|---|---|
| MF capsules (comparison) | 1 |
| Capsules according to PCT/EP2020/085804 | 3 |
| (comparison) | |
| Capsules with emulsion stabilizer according to | 2 |
| the description (comparison) | |
| Capsules with emulsion stabilizer according to | 1 |
| the description and emulsifier (according to the | |
| invention) | |

Capsule slurry

| | |
|---|---|
| MF capsules (comparison) | 3 |
| Capsules according to PCT/EP2020/085804 | 3 |
| (comparison) | |
| Capsules with emulsion stabilizer according to | 3 |
| the description (comparison) | |
| Capsules with emulsion stabilizer according to | 2 |
| the description and emulsifier (according to the | |
| invention) | |

The results show that an improved phase stability of the microcapsules can be achieved by using the emulsifier according to the invention both in the capsule slurry and in the end product.

### Example 4—Stability of Alternative Microcapsule Slurry with Pre-Dilution

In this example, the use of the emulsifier, i.e. the ethoxylated, hydrogenated castor oil, is omitted.

25 g of microcapsule slurry were placed in a 100 mL beaker and fitted with a magnetic stirring bar. While stirring at 600 rpm, a dilution with 25 g tap water (T=60° C.) was carried out. The water was added in a thin stream and then homogenized for approximately 1 min. The diluted mixture was added to the fabric softener base while still warm.

To incorporate the slurry into the fabric softener base, 45.83 g of fabric softener base was placed in a 100 mL beaker and fitted with a magnetic stirring bar. A pipette was then wetted with the corresponding slurry in advance to reduce the weighing error and the corresponding mass of slurry (4.17 g in total) was slowly dripped in while stirring at 550 rpm.

To evaluate the phase stability of the microcapsule slurry, the pre-diluted microcapsule slurry and a microcapsule slurry were incorporated into a fabric softener formulation according to the invention and either provided with an emulsifier or pre-diluted and stored for 4 weeks at room temperature (20-25° C.). The stability was evaluated on the following scale: 1=no phase separation, 2=slight phase separation, 3=medium phase separation, 4=strong phase separation and 5=very strong phase separation. The results are shown in Table 5 below.

TABLE 5
Phase stability of the capsules after incorporation into the end
product and after 4 weeks of storage at room temperature

| | Phase stability | |
|---|---|---|
| | of the after | Phase stability |
| Fabric softener | incorporation | after 4 weeks |
| (1 wt. % capsule slurry each, | of the capsule | at room |
| based on oven-dry slurry) | slurry | temperature |
| Capsules with emulsion stabilizer | 2 | 2 |
| according to the description without | | |
| emulsifier (comparison) | | |
| Capsules with emulsion stabilizer | 1 | 2 |
| according to the description without | | |
| emulsifier, pre-dilution (1:1) with | | |
| 60° C. water | | |
| Capsules with emulsion stabilizer | 1 | 1 |
| according to the description and | | |
| emulsifier (according to the | | |
| invention) | | |

Regarding further advantageous embodiments of the device according to the invention, reference is made to the general part of the description and the attached claims to avoid repetition.

Finally, it is expressly noted that the exemplary embodiments of the means and uses according to the invention described above serve only to explain the claimed subject matter and do not limit it to the embodiments described.

1. A microcapsule dispersion containing:
(a) biodegradable microcapsules comprising a core material and a shell, wherein the shell consists of at least one barrier layer and one stability layer, wherein the barrier layer surrounds the core material, wherein the stability layer comprises at least one biopolymer and is arranged on the outer surface of the barrier layer, and wherein optionally an emulsion stabilizer is arranged at the transition from the barrier layer to the stability layer; and
(b) at least one emulsifier, wherein the emulsifier is selected from the group of ethoxylated, hydrogenated castor oils, particularly those with medium EO values in the range of 20 to 60, preferably 30 to 50.

2. The microcapsule dispersion according to claim 1, wherein the proportion of the emulsifier based on the total weight of the microcapsule dispersion is 0.5 wt. % to 50 wt. %, preferably 1.0 wt. % to 30 wt. %, more preferably 2 wt. % to 20 wt. %, and most preferably 4 wt. % to 8 wt. %

3. The microcapsule dispersion according to claim 1, wherein the dispersion comprises the microcapsules as a solid phase and water as the main component of a continuous phase.

4. The microcapsule dispersion according to claim 3, wherein the emulsifier is a component of the continuous phase in which the microcapsules are dispersed.

5. The microcapsule dispersion according to claim 3, wherein the continuous phase consists of more than 50 wt. %, preferably more than 60 wt. %, more than 70 wt. %, or more than 80 wt. % water.

6. The microcapsule dispersion according to claim 3, containing at least one emulsifier in an amount of up to 40 wt. %, preferably up to 30 wt. % or up to 20 wt. %, more preferably up to a maximum of 10 wt. %, and most preferably in an amount of 2 wt. % to 10 wt. %

7. The microcapsule dispersion according to claim 3, wherein the continuous phase, based on the total weight of the continuous phase, contains 60 wt. % to 95 wt. %, preferably 70 wt. % to 95 wt. %, of water and 2 wt. % to 40 wt. %, preferably 2 wt. % to 20 wt. %, of the at least one emulsifier.

8. The microcapsule dispersion according to claim 3, wherein the microcapsule dispersion contains the microcapsules in an amount of 1 wt. % to 60 wt. %, preferably 15 wt. % to 50 wt. %, and particularly 20 wt. % to 35 wt. %.

9. The microcapsule dispersion according to claim 1, wherein the at least one emulsifier comprises or consists of PEG-40 hydrogenated Castor Oil (INCI).

10. A microcapsule composition obtained by drying the microcapsule dispersion according to claim 1, wherein the dried microcapsule composition contains less than 5 wt. %, preferably less than 1 wt. %, and most preferably no water except for unavoidable traces.

11. Use of the microcapsule dispersion according to claim 1 for the production of a product, wherein the microcapsule dispersion is used to produce the product or its intermediate, and wherein the final product or the intermediate product has a pH value of less than 11, preferably less than 9, more preferably less than 5, and most preferably less than 4 and/or a conductivity of at least 0.1 mS/cm, preferably at least 0.2 mS/cm, and at most 100 mS/cm, preferably up to 60 mS/cm, and most preferably 34 mS/cm.

12. A product containing a microcapsule dispersion according to claim 1, with a pH value of less than 11, preferably less than 9, more preferably less than 5, and most preferably less than 4 and/or a conductivity of at least 0.1 mS/cm, preferably at least 0.2 mS/cm, and at most 100 mS/cm, preferably up to 60 mS/cm, and most preferably 34 mS/cm.

13. The product according to claim 12, wherein the proportion of the microcapsule dispersion, based on the total weight of the product, is at least 0.1 wt. %, preferably at least 0.5 wt. % and preferably the proportion of the emulsifier in the product based on the total weight of the product is 0.001 wt. % to 0.25 wt. %, preferably 0.001 wt. % to 0.15 wt. %, more preferably 0.001 wt. % to 0.08 wt. %, and wherein the product is preferably liquid.

14. The product according to claim 12, wherein the product is selected from the group consisting of an adhesive system; a pharmaceutical product; a coating material, particularly coated paper; a thermal storage coating, a self-healing coating or a corrosion protection coating and such microcapsule-containing coatings for functional packaging materials.
